SEQUENCE LISTING (64 sequences)

SEQ ID NO: 1 - DNA, 351 bp, Artificial Sequence
  Description of Artificial Sequence Synthetic polynucleotide
gaggtgcagctggtggagagcggaggaggcctggtgcagccaggcagaagcctgaggctgtcttgcgccgcttccggcttcacctttgacgatcacgccatgcattgggtgcgtcaagcacccggcaagggcctggagtgggtgtctggcatctcctggaactccggcagcatcggctacgccgagtctgtgaagggcaggttcaccatcagccgggacaacgctaagaattctctgtatctgcaaatgaattccctgcgcgccgaggatacagctctgtactattgtgccaaggacaggcggggcccatttgattactggggccagggcaccctggtgacagtctcgagt

SEQ ID NO: 2 - DNA, 339 bp, Artificial Sequence
  Description of Artificial Sequence Synthetic polynucleotide
gacatcgtgatgacccagagccctgattctctggccgtgtctctgggcgagagagctacaatcaactgcaagtccagccagtccgtgctgtcttccagcaacaataagaattacctggcctggtatcagcagaagccaggccagccccctaagctgctgatctactgggcttccaccagggagagcggagtgccagaccggttcagcggctctggctccggcacagacttcaccctgacaatctcttccctgcaagccgaggacgtggccgtgtactattgtcagcagtactattccaccccacgcacattcggccagggcaccaaggtggagatcaag

SEQ ID NO: 3 - protein, 117 aa, Artificial Sequence
  Description of Artificial Sequence Synthetic polypeptide
EVQLVESGGGLVQPGRSLRLSCAASGFTFDDHAMHWVRQAPGKGLEWVSGISWNSGSIGYAESVKGRFTISRDNAKNSLYLQMNSLRAEDTALYYCAKDRRGPFDYWGQGTLVTVSS

SEQ ID NO: 4 - protein, 113 aa, Artificial Sequence
  Description of Artificial Sequence Synthetic polypeptide
DIVMTQSPDSLAVSLGERATINCKSSQSVLSSSNNKNYLAWYQQKPGQPPKLLIYWASTRESGVPDRFSGSGSGTDFTLTISSLQAEDVAVYYCQQYYSTPRTFGQGTKVEIK

SEQ ID NO: 5 - protein, 8 aa, Artificial Sequence
  Description of Artificial Sequence Synthetic peptide
GFTFDDHA

SEQ ID NO: 6 - protein, 8 aa, Artificial Sequence
  Description of Artificial Sequence Synthetic peptide
ISWNSGSI

SEQ ID NO: 7 - protein, 12 aa, Artificial Sequence
  Description of Artificial Sequence Synthetic peptide
CAKDRRGPFDYW

SEQ ID NO: 8 - protein, 12 aa, Artificial Sequence
  Description of Artificial Sequence Synthetic peptide
QSVLSSSNNKNY

SEQ ID NO: 9 - protein, 3 aa, Artificial Sequence
  Description of Artificial Sequence Synthetic peptide
WAS

SEQ ID NO: 10 - protein, 11 aa, Artificial Sequence [sequence omitted]

SEQ ID NO: 11 - DNA, 345 bp, Artificial Sequence
  Description of Artificial Sequence Synthetic polynucleotide
caggtgcagctggtggagtccggaggaggagtggtgcagccaggcaggtccctgaggctgagctgcgccgcttctggcttcaccttttccatctacggaatgcactgggtgaggcaggctcctggcaagggcctggagtgggtggctgtgatctcctatgacggcagcaacaagtactatgccgatagcgtgaagggcagattcaccatctctcgcgacaactccaagaatacactgtacctgcagatgaactctctgagagccgaggacacagccgtgtactattgtgccgctggcaattggtttgatccatggggccagggcaccctggtgacagtctcgagt

SEQ ID NO: 12 - DNA, 321 bp, Artificial Sequence
  Description of Artificial Sequence Synthetic polynucleotide
gacatccagctgacacagagcccttctttcctgtccgcctccgtgggcgatagggtgaccatcacatgccgggcctctcagggcatctccagctacctggcttggtatcagcagaagcccggcaaggcccctaagctgctgatctacgctgcttctaccctgcagtccggagtgccaagcagattctctggctccggcagcggcacagagtttaccctgacaatctcttccctgcaaccagaggacttcgccacctactattgtcagcagctgaaccgctatcccctgacctttggcggcggcacaaaggtggagatcaag

SEQ ID NO: 13 - protein, 115 aa, Artificial Sequence
  Description of Artificial Sequence Synthetic polypeptide
QVQLVESGGGVVQPGRSLRLSCAASGFTFSIYGMHWVRQAPGKGLEWVAVISYDGSNKYYADSVKGRFTISRDNSKNTLYLQMNSLRAEDTAVYYCAAGNWFDPWGQGTLVTVSS

SEQ ID NO: 14 - protein, 107 aa, Artificial Sequence
  Description of Artificial Sequence Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNRYPLTFGGGTKVEIK

SEQ ID NO: 15 - protein, 8 aa, Artificial Sequence
  Description of Artificial Sequence Synthetic peptide
GFTFSIYG

SEQ ID NO: 16 - protein, 8 aa, Artificial Sequence
  Description of Artificial Sequence Synthetic peptide
ISYDGSNK

SEQ ID NO: 17 - protein, 10 aa, Artificial Sequence
  Description of Artificial Sequence Synthetic peptide
CAAGNWFDPW

SEQ ID NO: 18 - protein, 6 aa, Artificial Sequence
  Description of Artificial Sequence Synthetic peptide
QGISSY

SEQ ID NO: 19 - protein, 3 aa, Artificial Sequence
  Description of Artificial Sequence Synthetic peptide
AAS

SEQ ID NO: 20 - protein, 11 aa, Artificial Sequence [sequence omitted]

SEQ ID NO: 21 - DNA, 363 bp, Artificial Sequence
  Description of Artificial Sequence Synthetic polynucleotide
caggtgcagctggtggagagcggaggaggagtggtgcagccaggcaggtccctgaggctgagctgcgccgcttctggcttcaccttttctaactacgccatgcactgggtgaggcaggctcctggcaagggcctggagtgggtggccctgatctccttcgacggcagctataagtactatgctgattctgtgaagggcaggttcaccatctcccgcgacaactcaaagaatacactgtacctgcagatgaattccctgagagccgaggacacagccgtgtactattgcgtgaaggatgccggctactccagctcttggttctttgattattggggccagggcaccctggtgacagtctcgagt

SEQ ID NO: 22 - DNA, 321 bp, Artificial Sequence
  Description of Artificial Sequence Synthetic polynucleotide
gacatccagatgacccagtccccttccagcgtgtctgcttccgtgggcgacagggtgaccatcacatgccgggccagccaggatatctcttcctggctggcttggtaccagcagaagcccggcaaggcccctaagctgctgatctatgctgcctcctccctgcagagcggagtgccatctaggttcagcggctctggctccggcacagactttaccctgacaatctcctcactgcagccagaggatttcggcaactactattgtcagcaggccaattctttcccctggacctttggccagggcacaaaggtggagatcaag

SEQ ID NO: 23 - protein, 121 aa, Artificial Sequence
  Description of Artificial Sequence Synthetic polypeptide
QVQLVESGGGVVQPGRSLRLSCAASGFTFSNYAMHWVRQAPGKGLEWVALISFDGSYKYYADSVKGRFTISRDNSKNTLYLQMNSLRAEDTAVYYCVKDAGYSSSWFFDYWGQGTLVTVSS

SEQ ID NO: 24 - protein, 107 aa, Artificial Sequence
  Description of Artificial Sequence Synthetic polypeptide
DIQMTQSPSSVSASVGDRVTITCRASQDISSWLAWYQQKPGKAPKLLIYAASSLQSGVPSRFSGSGSGTDFTLTISSLQPEDFGNYYCQQANSFPWTFGQGTKVEIK

SEQ ID NO: 25 - protein, 8 aa, Artificial Sequence
  Description of Artificial Sequence Synthetic peptide
GFTFSNYA

SEQ ID NO: 26 - protein, 8 aa, Artificial Sequence
  Description of Artificial Sequence Synthetic peptide
ISFDGSYK

SEQ ID NO: 27 - protein, 16 aa, Artificial Sequence
  Description of Artificial Sequence Synthetic peptide
CVKDAGYSSSWFFDYW

SEQ ID NO: 28 - protein, 6 aa, Artificial Sequence
  Description of Artificial Sequence Synthetic peptide
QDISSW

SEQ ID NO: 29 - protein, 3 aa, Artificial Sequence
  Description of Artificial Sequence Synthetic peptide
AAS

SEQ ID NO: 30 - protein, 11 aa, Artificial Sequence [sequence omitted]

SEQ ID NO: 31 - DNA, 363 bp, Artificial Sequence
  Description of Artificial Sequence Synthetic polynucleotide
gaggtgcagctggtggagtccggaggaggcctggtgcagccaggaggctccctgaggctgagctgcgccgcttctggcttcacctttacatcctactggatgagctgggtgagacaggctccaggcaagggcctggagtgggtggctaacatcaagcaggacggctctgagaagtactatgtggattccgtgaagggcaggttcaccatcagccgggacaacgccaagaattctctgtatctgcaaatgaattccctgagagccgaggatacagccgtgtactattgtgcccgcggcccagtggctggaccttactggtattttgacctgtggggcaggggcaccctggtgacagtctcgagt

SEQ ID NO: 32 - DNA, 321 bp, Artificial Sequence
  Description of Artificial Sequence Synthetic polynucleotide
gccatccagatgacccagagcccttccagcctgtccgcctccgtgggcgacagggtgaccatcacatgccgggcctctcagggcatcagaaacgatctgggctggtaccagcagaagcccggcaaggctcctaagctgctgatctatgccgcttcttccctgcaatctggagtgccatcccgcttctctggatccggaagcggaaccgactttaccctgacaatcagctctctgcagccagaggacttcgccacatactattgtctgcaggattaccactatcccctgacctttggcggcggcacaaaggtggagatcaag

SEQ ID NO: 33 - protein, 121 aa, Artificial Sequence
  Description of Artificial Sequence Synthetic polypeptide
EVQLVESGGGLVQPGGSLRLSCAASGFTFTSYWMSWVRQAPGKGLEWVANIKQDGSEKYYVDSVKGRFTISRDNAKNSLYLQMNSLRAEDTAVYYCARGPVAGPYWYFDLWGRGTLVTVSS

SEQ ID NO: 34 - protein, 107 aa, Artificial Sequence
  Description of Artificial Sequence Synthetic polypeptide
AIQMTQSPSSLSASVGDRVTITCRASQGIRNDLGWYQQKPGKAPKLLIYAASSLQSGVPSRFSGSGSGTDFTLTISSLQPEDFATYYCLQDYHYPLTFGGGTKVEIK

SEQ ID NO: 35 - protein, 8 aa, Artificial Sequence
  Description of Artificial Sequence Synthetic peptide
GFTFTSYW

SEQ ID NO: 36 - protein, 8 aa, Artificial Sequence
  Description of Artificial Sequence Synthetic peptide
IKQDGSEK

SEQ ID NO: 37 - protein, 16 aa, Artificial Sequence
  Description of Artificial Sequence Synthetic peptide
CARGPVAGPYWYFDLW

SEQ ID NO: 38 - protein, 6 aa, Artificial Sequence
  Description of Artificial Sequence Synthetic peptide
QGIRND

SEQ ID NO: 39 - protein, 3 aa, Artificial Sequence
  Description of Artificial Sequence Synthetic peptide
AAS

SEQ ID NO: 40 - protein, 11 aa, Artificial Sequence
  Description of Artificial Sequence Synthetic peptide
CLQDYHYPLTF

SEQ ID NO: 41 - DNA, 375 bp, Artificial Sequence
  Description of Artificial Sequence Synthetic polynucleotide
caggtgcagctgcagcagtccggaccaggcctggtgaagccttctcagaccctgtccctgacatgcgccatcagcggcgactccgtgtcctccaactccgccacctggaattggatcagacagtctccttcccgcggcctggagtggctgggaaggacatactatcggtctaagtggtacaacgactatgccgtgtccgtgaagagcaggatcaccatcaacccagatacaagcaagaatcagttctctctgcagctgaattccgtgacccccgaggacacagccgtgtactattgtgctagaggcggacactccatcgctgtggctggaccatttgattactggggccagggcaccctggtgacagtctcgagt

SEQ ID NO: 42 - DNA, 342 bp, Artificial Sequence
  Description of Artificial Sequence Synthetic polynucleotide
gacatcgtgatgacccagtcccctgatagcctggccgtgagcctgggagagagggctacaatcaactgcaagtccagccagtctgtgctgtactcttccaacaataagaattacctggcctggtatcagcagaagccaggccagccccctaagctgctgatctattgggcttctaccagggactccggagtgccagatcggttctccggaagcggatctggcacagactttaccctgacaatcagctctctgcaagccgaggatgtggccgtgtactattgtcagcagtactataactccccaccacctaccttcggccagggaacaaaggtggagatcaag

SEQ ID NO: 43 - protein, 125 aa, Artificial Sequence
  Description of Artificial Sequence Synthetic polypeptide
QVQLQQSGPGLVKPSQTLSLTCAISGDSVSSNSATWNWIRQSPSRGLEWLGRTYYRSKWYNDYAVSVKSRITINPDTSKNQFSLQLNSVTPEDTAVYYCARGGHSIAVAGPFDYWGQGTLVTVSS

SEQ ID NO: 44 - protein, 114 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRDSGVPDRFSGSGSGTDFTLTISSLQAEDVAVYYCQQYYNSPPPTFGQGTKVEIK

SEQ ID NO: 45 - protein, 10 aa, Artificial Sequence
  Description of Artificial Sequence Synthetic peptide
GDSVSSNSAT

SEQ ID NO: 46 - protein, 7 aa, Artificial Sequence
  Description of Artificial Sequence Synthetic peptide
TYYRSKW

SEQ ID NO: 47 - protein, 17 aa, Artificial Sequence
  Description of Artificial Sequence Synthetic peptide
CARGGHSIAVAGPFDYW

SEQ ID NO: 48 - protein, 12 aa, Artificial Sequence
  Description of Artificial Sequence Synthetic peptide
QSVLYSSNNKNY

SEQ ID NO: 49 - protein, 3 aa, Artificial Sequence
  Description of Artificial Sequence Synthetic peptide
WAS

SEQ ID NO: 50 - protein, 12 aa, Artificial Sequence [sequence omitted]

SEQ ID NO: 51 - DNA, 366 bp, Artificial Sequence
  Description of Artificial Sequence Synthetic polynucleotide
caggtgcagctggtggagtctggaggaggagtggtgcagccaggcaggtccctgaggctgtcctgcgccgcctccggcttcaccttttccatctacggaatgcactgggtgaggcaggctccaggcaagggcctggagtgggtggctgtgatctcttatgacggctccaacaagtactatgccgattccgtgaagggcagattcaccatcagccgcgacaactctaagaatacactgtacctgcagatgaatagcctgagagccgaggatacagccgtgtactattgtgctaaggaccccgattactatggctccggcagctactatcctgactggggccagggcaccctggtgacagtctcgagt

SEQ ID NO: 52 - DNA, 321 bp, Artificial Sequence
  Description of Artificial Sequence Synthetic polynucleotide
gacatccagctgacacagagcccttctttcctgtccgcctccgtgggcgatagggtgaccatcacatgccgggcctctcagggcatctccagctacctggcttggtatcagcagaagcccggcaaggcccctaagctgctgatctacgctgcttctaccctgcagtccggagtgccaagcaggttctctggctccggcagcggcacagagtttaccctgacaatctcttccctgcaaccagaggacttcgccacctactattgtcagcaggtgaagtcctatcccctgacctttggcggcggcacaaaggtggagatcaag

SEQ ID NO: 53 - protein, 122 aa, Artificial Sequence
  Description of Artificial Sequence Synthetic polypeptide
QVQLVESGGGVVQPGRSLRLSCAASGFTFSIYGMHWVRQAPGKGLEWVAVISYDGSNKYYADSVKGRFTISRDNSKNTLYLQMNSLRAEDTAVYYCAKDPDYYGSGSYYPDWGQGTLVTVSS

SEQ ID NO: 54 - protein, 107 aa, Artificial Sequence
  Description of Artificial Sequence Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQVKSYPLTFGGGTKVEIK

SEQ ID NO: 55 - protein, 8 aa, Artificial Sequence
  Description of Artificial Sequence Synthetic peptide
GFTFSIYG

SEQ ID NO: 56 - protein, 8 aa, Artificial Sequence
  Description of Artificial Sequence Synthetic peptide
ISYDGSNK

SEQ ID NO: 57 - protein, 17 aa, Artificial Sequence
  Description of Artificial Sequence Synthetic peptide
CAKDPDYYGSGSYYPDW

SEQ ID NO: 58 - protein, 6 aa, Artificial Sequence
  Description of Artificial Sequence Synthetic peptide
QGISSY

SEQ ID NO: 59 - protein, 3 aa, Artificial Sequence
  Description of Artificial Sequence Synthetic peptide
AAS

SEQ ID NO: 60 - protein, 11 aa, Artificial Sequence [sequence omitted]

SEQ ID NO: 61 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 107 aa, Artificial Sequence [sequence omitted]

SEQ ID NO: 63 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 64 - protein, 15 aa, Artificial Sequence
  Description of Artificial Sequence Synthetic peptide
GGGGSGGGGSGGGGS

(19) United States
(12) Patent Application Publication
(10) Pub. No.: US 2021/0214454 A1
(43) Pub. Date: Jul. 15, 2021

(54) ANTI-CD40 ANTIBODIES AND COMPOSITIONS

(71) Applicant: SYMPHOGEN A/S, Ballerup (DK)

(72) Inventors: Anton WILLER, Virum (DK); Trine LINDSTED, Farum (DK); Klaus KOEFOED, Copenhagen S (DK); Mikkel Wandahl PEDERSEN, Allerød (DK); Lise Camilla FRÖHLICH, København Ø (DK)

(73) Assignee: SYMPHOGEN A/S, Ballerup (DK)

(21) Appl. No.: 17/144,584

(22) Filed: Jan. 8, 2021

Related U.S. Application Data
(60) Provisional application No. 62/959,598, filed on Jan. 10, 2020.

(51) Int. Cl. C07K 16/28; A61P 35/00

(52) U.S. Cl. CPC C07K 16/2878; A61P 35/00; A61K 2039/505; C07K 2317/31; C07K 2317/74; C07K 2317/92

(57) ABSTRACT

This invention relates to anti-CD40 antibodies and methods of using them in treating diseases and conditions related to CD40 activity, e.g., cancer.

### CROSS-REFERENCE TO RELATED APPLICATION

This application claims priority from U.S. Provisional Patent Application 62/959,598, filed Jan. 10, 2020, whose disclosure is incorporated by reference herein in its entirety.

### SEQUENCE LISTING

The instant application contains a Sequence Listing which has been submitted electronically in ASCII format and is hereby incorporated by reference in its entirety. The electronic copy of the Sequence Listing, created on Dec. 22, 2020, is named 022675_US061_SL.txt and is 36,450 bytes in size.

### BACKGROUND OF THE INVENTION

CD40 is a costimulatory transmembrane protein that is a member of the TNF receptor superfamily. CD40 is expressed on antigen-presenting immune cells, such as B cells, dendritic cells (DCs), monocytes and macrophages, and is central to many immune and inflammatory responses. CD40 also is expressed on various types of tumor cells, including most B cell malignancies (e.g., multiple myeloma, non-Hodgkin's lymphoma, and chronic lymphocytic leukemia) and many solid tumors.

CD40L, the ligand for CD40, is expressed on activated T cells and platelets. The CD40:CD40L interaction promotes clustering of CD40, which is required for strong induction of intracellular signaling and activation. Binding of CD40L to CD40 on the surface of antigen-presenting cells stimulates their activation and production of proinflammatory cytokines, and the CD40:CD40L interaction is necessary for successful adaptive immune responses. On dendritic cells, activation of CD40 increases cell-surface expression of costimulatory and MHC molecules as well as inducing proinflammatory cytokines, leading to enhanced B and T cell activation. Activation of CD40 on B cells is important for generation of long-lived plasma cells and memory B cells and induces proliferation, immunoglobulin class switching, and antibody secretion. On macrophages infiltrating tumor tissues or populated in the microenvironment of solid tumors, binding of CD40L to CD40 drives phenotype switching from M2 to M1 type, which is accompanied by induction of cytotoxic T cell and natural killer (NK) cell responses against tumors. Thus, CD40 plays a central role in regulation of the adaptive immune system and can induce antitumor effects through activation of several cell types.

In view of the critical role of CD40 in regulating the immune response, there is a need for new and improved therapies that target CD40 to treat cancer and immune disorders.

### SUMMARY OF THE INVENTION

The present invention is directed to novel recombinant antibodies targeting CD40, as well as pharmaceutical compositions comprising one or more (e.g., two) of these antibodies, and use of the antibodies and pharmaceutical compositions for enhancing immune activity and treating cancer. The antibodies and compositions described herein may be used in a method for enhancing immune activity or treating cancer or an immune disorder in a patient; may be used for the manufacture of a medicament for enhancing immune activity or treating cancer or an immune disorder in a patient; or may be for use in enhancing immune activity or treating cancer or an immune disorder in a patient. Compared to currently available treatments for cancers, including antibody treatments, it is contemplated that the antibodies and compositions described herein may provide a superior clinical response either alone or in combination with another cancer therapeutic.

In some embodiments, the present disclosure provides an anti-CD40 antibody or an antigen-binding portion thereof that competes or cross-competes for binding with or binds to the same epitope of human CD40 as antibody 17303, 16040, 15833, 16154, 15888, or 15948. In certain embodiments, the anti-CD40 antibody or antigen-binding portion is defined by the amino acid sequences of the six CDRs, heavy and light chain variable domains, or heavy and light chains of said antibody.

In some embodiments, the present disclosure provides an anti-CD40 antibody or an antigen-binding portion thereof, wherein
a) the heavy chain of said antibody comprises:
   i) heavy chain complementarity determining regions (H-CDR)-1-3 comprising the amino acid sequences of SEQ ID NOs: 5-7, respectively;
   ii) a heavy chain variable domain (VH) comprising an amino acid sequence at least 90% identical to the amino acid sequence of SEQ ID NO: 3;
   iii) a VH comprising the amino acid sequence of SEQ ID NO: 3; or
   iv) a heavy chain (HC) comprising the amino acid sequences of SEQ ID NOs: 3 and 61; and
b) the light chain of said antibody comprises:
   i) light chain complementarity determining regions (L-CDR)-1-3 comprising the amino acid sequences of SEQ ID NOs: 8-10, respectively;
   ii) a light chain variable domain (VL) comprising an amino acid sequence at least 90% identical to the amino acid sequence of SEQ ID NO: 4;
   iii) a VL comprising the amino acid sequence of SEQ ID NO: 4; or
   iv) a light chain (LC) comprising the amino acid sequences of SEQ ID NOs: 4 and 62.

In some embodiments, the present disclosure provides an anti-CD40 antibody or an antigen-binding portion thereof, wherein
a) the heavy chain of said antibody comprises:
   i) H-CDR-1-3 comprising the amino acid sequences of SEQ ID NOs: 15-17, respectively;
   ii) a VH comprising an amino acid sequence at least 90% identical to the amino acid sequence of SEQ ID NO: 13;
   iii) a VH comprising the amino acid sequence of SEQ ID NO: 13; or
   iv) an HC comprising the amino acid sequences of SEQ ID NOs: 13 and 61; and
b) the light chain of said antibody comprises:
   i) L-CDR-1-3 comprising the amino acid sequences of SEQ ID NOs: 18-20, respectively;
   ii) a VL comprising an amino acid sequence at least 90% identical to the amino acid sequence of SEQ ID NO: 14;
   iii) a VL comprising the amino acid sequence of SEQ ID NO: 14; or
   iv) an LC comprising the amino acid sequences of SEQ ID NOs: 14 and 62.

In some embodiments, the present disclosure provides an anti-CD40 antibody or an antigen-binding portion thereof, wherein
a) the heavy chain of said antibody comprises:
   i) H-CDR-1-3 comprising the amino acid sequences of SEQ ID NOs: 25-27, respectively;
   ii) a VH comprising an amino acid sequence at least 90% identical to the amino acid sequence of SEQ ID NO: 23;
   iii) a VH comprising the amino acid sequence of SEQ ID NO: 23; or
   iv) an HC comprising the amino acid sequences of SEQ ID NOs: 23 and 61; and
b) the light chain of said antibody comprises:
   i) L-CDR-1-3 comprising the amino acid sequences of SEQ ID NOs: 28-30, respectively;
   ii) a VL comprising an amino acid sequence at least 90% identical to the amino acid sequence of SEQ ID NO: 24;
   iii) a VL comprising the amino acid sequence of SEQ ID NO: 24; or
   iv) an LC comprising the amino acid sequences of SEQ ID NOs: 24 and 62.

In some embodiments, the present disclosure provides an anti-CD40 antibody or an antigen-binding portion thereof, wherein
a) the heavy chain of said antibody comprises:
   i) H-CDR-1-3 comprising the amino acid sequences of SEQ ID NOs: 35-37, respectively;
   ii) a VH comprising an amino acid sequence at least 90% identical to the amino acid sequence of SEQ ID NO: 33;
   iii) a VH comprising the amino acid sequence of SEQ ID NO: 33; or
   iv) an HC comprising the amino acid sequences of SEQ ID NOs: 33 and 61; and
b) the light chain of said antibody comprises:
   i) L-CDR-1-3 comprising the amino acid sequences of SEQ ID NOs: 38-40, respectively;
   ii) a VL comprising an amino acid sequence at least 90% identical to the amino acid sequence of SEQ ID NO: 34;
   iii) a VL comprising the amino acid sequence of SEQ ID NO: 34; or
   iv) an LC comprising the amino acid sequences of SEQ ID NOs: 34 and 62.

In some embodiments, the present disclosure provides an anti-CD40 antibody or an antigen-binding portion thereof, wherein
a) the heavy chain of said antibody comprises:
   i) H-CDR-1-3 comprising the amino acid sequences of SEQ ID NOs: 45-47, respectively;
   ii) a VH comprising an amino acid sequence at least 90% identical to the amino acid sequence of SEQ ID NO: 43;
   iii) a VH comprising the amino acid sequence of SEQ ID NO: 43; or
   iv) an HC comprising the amino acid sequences of SEQ ID NOs: 43 and 61; and
b) the light chain of said antibody comprises:
   i) L-CDR-1-3 comprising the amino acid sequences of SEQ ID NOs: 48-50, respectively;
   ii) a VL comprising an amino acid sequence at least 90% identical to the amino acid sequence of SEQ ID NO: 44;
   iii) a VL comprising the amino acid sequence of SEQ ID NO: 44; or
   iv) an LC comprising the amino acid sequences of SEQ ID NOs: 44 and 62.

In some embodiments, the present disclosure provides an anti-CD40 antibody or an antigen-binding portion thereof, wherein
a) the heavy chain of said antibody comprises:
   i) H-CDR-1-3 comprising the amino acid sequences of SEQ ID NOs: 55-57, respectively;
   ii) a VH comprising an amino acid sequence at least 90% identical to the amino acid sequence of SEQ ID NO: 53;
   iii) a VH comprising the amino acid sequence of SEQ ID NO: 53; or
   iv) an HC comprising the amino acid sequences of SEQ ID NOs: 53 and 61; and
b) the light chain of said antibody comprises:
   i) L-CDR-1-3 comprising the amino acid sequences of SEQ ID NOs: 58-60, respectively;
   ii) a VL comprising an amino acid sequence at least 90% identical to the amino acid sequence of SEQ ID NO: 54;
   iii) a VL comprising the amino acid sequence of SEQ ID NO: 54; or
   iv) an LC comprising the amino acid sequences of SEQ ID NOs: 54 and 62.

The present disclosure also provides isolated nucleic acid molecules, vectors, and host cells comprising nucleotide sequences that encode the heavy chain or an antigen-binding portion thereof, the light chain or an antigen-binding portion thereof, or both, of an anti-CD40 antibody or antigen-binding portion described herein. Further, the present disclosure provides methods for producing an anti-CD40 or antigen-binding portion described herein by culturing said host cells, as well as methods for producing an antibody composition by admixing antibodies or antigen-binding portions described herein.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE DRAWINGS

FIGS. 1A-1C are a set of graphs showing the binding of different anti-CD40 antibodies to human (FIG. 1A) and cynomolgus (FIG. 1B) CD40 expressed on CHO-S cells. Mock transfected CHO-S cells were used as a negative control (FIG. 1C). Data are presented as mean±SEM.

FIG. 2 is a graph showing the activity of single antibodies and antibody mixtures in a CD40-NFkB-luciferase reporter assay. Data are normalized to untreated controls and presented as a single XY data point, with values from replicate 1 on the X-axis and values from replicate 2 on the Y-axis.

FIG. 3 is a graph showing the activity of single antibodies and antibody mixtures in a B cell proliferation assay. Data are normalized to untreated controls and presented as a single XY data point, with values from replicate 1 on the X-axis and values from replicate 2 on the Y-axis.

FIG. 4 is a pair of graphs showing dose-response relationships of single mAbs (top panel) and mixtures (bottom panel) in a CD40-NFkB-luciferase assay. Data are normalized to untreated controls and presented as mean±SEM.

FIG. 5 is a graph showing the activity of single antibodies and antibody mixtures in a CD40-NFkB-luciferase assay without CD40 ligand (grey) and in the presence of CD40 ligand (black). Data are presented as mean±SEM.

FIG. 6 is a graph showing dose-response relationships of anti-CD40 antibody mixtures in a one-way MLR assay. Data are normalized to untreated controls and presented as mean±SEM.

FIG. 7 is a pair of graphs showing dose-response relationships of single mAbs (top panel) and mixtures (bottom panel) in a B cell proliferation assay. Data are normalized to untreated controls and presented as mean±SEM.

FIG. 8 is a pair of graphs showing dose-response relationships of single antibodies (top panel) and mixtures (bottom panel) in a dendritic cell activation assay. Data are normalized to untreated controls and presented as mean±SEM.

FIG. 9 is a graph showing the dose-response relationship of anti-CD40 antibody mixture 17303+16040, selicrelumab analogue, and CD40L in a B cell proliferation assay using B cells isolated from CD40 HuGEMM mice. Data are normalized to untreated controls and presented as mean±SEM.

FIG. 10 is a graph showing tumor growth in huCD34-NOG mice subcutaneously engrafted with Raji tumor cells. The mice were treated twice weekly with the antibody mixture for a total of six doses. The grey area denotes the treatment period. Data are presented as mean±SEM. **p<0.01.

FIG. 11 is a pair of graphs showing tumor growth in CD40 HuGEMM mice subcutaneously engrafted with MC38-OVA murine tumor cells. The mice were treated with the antibody mixture or the selicrelumab analogue twice weekly for a total of six doses. Tumor volume (left panel) and body weight (right panel) were measured twice weekly. The grey area denotes the treatment period. Data are presented as mean±SEM.

FIG. 12 is a schematic showing competition patterns and epitope bins identified for the tested anti-CD40 antibodies. Connecting black lines indicate cross-blocking activity. Connecting dotted lines indicate blocking in one direction. Circles represent antibodies tested in both directions, and squares represent antibodies tested in only one direction. Antibodies are grouped according to competition patterns with other anti-CD40 antibodies.

FIG. 13 is a series of structures illustrating the binding epitopes of antibodies 17303, 16040, and 15833, and the selicrelumab analogue mapped on the structure of CD40 CRD1-3 (light grey) in complex with CD154 (white) (PDB: 3QD6). The structures are shown as a surface representation and viewed vertically with the N-terminus pointing upwards, or seen from the N-terminal top or C-terminal bottom as indicated. Linear epitopes are shown as dark grey and contact residues as black. A) Epitope of 17303, B), epitope of 16040, C) epitope of 15833, and D) epitope of selicrelumab analogue.

### DETAILED DESCRIPTION OF THE INVENTION

The present disclosure provides new anti-human CD40 antibodies that can be used to stimulate CD40 activity in a patient, such as a cancer patient. Unless otherwise stated, as used herein, “CD40” refers to human CD40. A human CD40 polypeptide sequence is available under UniProt Accession No. P25942 (TNRS_HUMAN) (SEQ ID NO: 63), as shown below:

```
                10 20 30 40
MVRLPLQCVL WGCLLTAVHP EPPTACREKQ YLINSQCCSL
                50 60 70 80
CQPGQKLVSD CTEFTETECL PCGESEFLDT WNRETHCHQH
                90 100 110 120
KYCDPNLGLR VQQKGTSETD TICTCEEGWH CTSEACESCV
              130 140 150 160
LHRSCSPGFG VKQIATGVSD TICEPCPVGF FSNVSSAFEK
              170 180 190 200
CHPWTSCETK DLVVQQAGTN KTDVVCGPQD RLRALVVIPI
              210 220 230 240
IFGILFAILL VLVFIKKVAK KPTNKAPHPK QEPQEINFPD
              250 260 270
DLPGSNTAAP VQETLHGCQP VTQEDGKESR ISVQERQ
```

The term “antibody” (Ab) or “immunoglobulin” (Ig), as used herein, refers to a tetramer comprising two heavy (H) chains (about 50-70 kDa) and two light (L) chains (about 25 kDa) inter-connected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. The assignment of amino acid numbers, and of FR and CDR regions, in the heavy or light chain may be in accordance with IMGT® definitions (Eu numbering; Lefranc et al., Dev Comp Immunol 27(1):55-77 (2003)); or the definitions of Kabat, Sequences of Proteins of Immunological Interest (National Institutes of Health, Bethesda, Md. (1987 and 1991)); Chothia & Lesk, J. Mol. Biol. 196:901-917 (1987); Chothia et al., Nature 342:878-883 (1989); MacCallum et al., J. Mol. Biol. 262:732-745 (1996); or Honegger and Plückthun, J. Mol. Biol. 309(3):657-70 (2001).

The term “recombinant antibody” refers to an antibody that is expressed from a cell or cell line comprising the nucleotide sequence(s) that encode the antibody, wherein said nucleotide sequence(s) are not naturally associated with the cell.

The term “isolated protein,” “isolated polypeptide” or “isolated antibody” refers to a protein, polypeptide or antibody that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “affinity” refers to a measure of the attraction between an antigen and an antibody. The intrinsic attractiveness of the antibody for the antigen is typically expressed as the binding affinity equilibrium constant (K_{D}) of a particular antibody-antigen interaction. An antibody is said to specifically bind to an antigen when the K_{D} is ≤1 mM, e.g., ≤1 uM, ≤100 nM, or ≤10 nM. A K_{D} binding affinity constant can be measured, e.g., by surface plasmon resonance (e.g., BIAcore™) using the IBIS MX96 SPR system from IBIS Technologies or the Carterra LSA SPR platform or by Bio-Layer Interferometry, for example using the Octet™ system from ForteBio.

The term “epitope” as used herein refers to a portion (determinant) of an antigen that specifically binds to an antibody or a related molecule such as a bi-specific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest (e.g., CD40) or a relevant portion thereof, then screening for binding to the epitope.

One can determine whether an antibody binds to the same epitope as or competes for binding with an anti-CD40 antibody of the present disclosure by using methods known in the art, including, without limitation, competition assays, epitope binning, and alanine scanning. In some embodiments, one allows the anti-CD40 antibody of the present disclosure to bind to CD40 under saturating conditions, and then measures the ability of the test antibody to bind to CD40. If the test antibody is able to bind to CD40 at the same time as the reference anti-CD40 antibody, then the test antibody binds to a different epitope than the reference anti-CD40 antibody. However, if the test antibody is not able to bind to CD40 at the same time, then the test antibody binds to the same epitope, an overlapping epitope, or an epitope that is in close proximity to the epitope bound by the anti-CD40 antibody of the present disclosure. This experiment can be performed using, e.g., ELISA, RIA, BIACORE™ SPR, Bio-Layer Interferometry or flow cytometry. To test whether an anti-CD40 antibody cross-competes with another anti-CD40 antibody, one may use the competition method described above in two directions, i.e., determining if the known antibody blocks the test antibody and vice versa. Such cross-competition experiments may be performed, e.g., using an IBIS MX96 or Carterra LSA SPR instrument or the Octet™ system.

The term “human antibody” refers to an antibody in which the variable domain and constant region sequences are derived from human sequences. The term encompasses antibodies with sequences that are derived from human genes but have been modified, e.g., to decrease immunogenicity, increase affinity, and/or increase stability. Further, the term encompasses antibodies produced recombinantly in nonhuman cells, which may impart glycosylation not typical of human cells. The term also encompasses antibodies produced in transgenic nonhuman organisms with human antibody genes (e.g., OmniRat® rats).

The term “antigen-binding portion” of an antibody (or simply “antibody portion”), as used herein, refers to one or more portions or fragments of an antibody that retain the ability to specifically bind to an antigen (e.g., human CD40, or a portion thereof). It has been shown that certain fragments of a full-length antibody can perform the antigen-binding function of the antibody. Examples of binding fragments encompassed within the term “antigen-binding portion” include (i) a Fab fragment: a monovalent fragment consisting of the VL, VH, CL and CH1 domains; (ii) a F(ab′)2 fragment: a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated complementarity determining region (CDR) capable of specifically binding to an antigen. Furthermore, although the two domains of the Fv fragment, VL and VH, are encoded by separate genes, they can be joined, using recombinant methods, by a synthetic linker that enables them to be made as a single protein chain in which the VL and VH domains pair to form monovalent molecules (known as single chain Fv (scFv)). Also within the present disclosure are antigen-binding molecules comprising a VH and/or a VL. In the case of a VH, the molecule may also comprise one or more of a CH1, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bi-specific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesin molecules can be obtained using standard recombinant DNA techniques, e.g., as described herein.

The class (isotype) and subclass of anti-CD40 antibodies may be determined by any method known in the art. In general, the class and subclass of an antibody may be determined using antibodies that are specific for a particular class and subclass of antibody. Such antibodies are available commercially. The class and subclass can be determined by ELISA or Western blot as well as other techniques. Alternatively, the class and subclass may be determined by sequencing all or a portion of the constant region of the heavy and/or light chains of the antibodies, comparing their amino acid sequences to the known amino acid sequences of various classes and subclasses of immunoglobulins, and determining the class and subclass of the antibodies.

Unless otherwise indicated, all antibody amino acid residue numbers referred to in this disclosure are those under the IMGT® numbering scheme (EU numbering).

### Anti-CD40 Antibodies

The present disclosure provides antibodies directed against CD40, and antigen-binding portions thereof. In some embodiments, the antibodies disclosed herein are human antibodies generated from transgenic animals (e.g., rats) that are able to produce antibodies encoded by rearranged human antibody genes. In certain embodiments, the human antibodies may contain certain mutations, e.g., to change primer-derived mutations back to the germline sequence (see, e.g., the “Symplex-corrected” variant sequences in Table 1).

Ligand induced clustering of CD40 is required for activation and induction of robust intracellular signaling. The majority of clinical stage anti-CD40 antibodies are dependent on Fc receptor interactions to achieve clustering and CD40 activation. However, Fc receptor interactions may cause liver and platelet toxicities as well as immune cell depletion, limiting the clinical effectiveness of anti-CD40 treatments. In some embodiments, the anti-CD40 antibodies of the present disclosure have the “LALA” mutations (L234A/L235A) in the Fc region. These mutations hinder the antibodies' binding to human FcγR (Fc gamma receptors). Such antibodies are advantageous because they have a low level of secondary effector functions and hence do not deplete effector T cells or target other non-malignant cells.

In some embodiments, the anti-CD40 antibody or antigen-binding portion competes or cross-competes for binding to human CD40 with, or binds to the same epitope of human CD40 as, an antibody comprising:
a) a heavy chain (HC) comprising the amino acid sequences of SEQ ID NOs: 3 and 61 and a light chain (LC) comprising the amino acid sequences of SEQ ID NOs: 4 and 62;
b) an HC comprising the amino acid sequences of SEQ ID NOs: 13 and 61 and an LC comprising the amino acid sequences of SEQ ID NOs: 14 and 62;
c) an HC comprising the amino acid sequences of SEQ ID NOs: 23 and 61 and an LC comprising the amino acid sequences of SEQ ID NOs: 24 and 62;
d) an HC comprising the amino acid sequences of SEQ ID NOs: 33 and 61 and an LC comprising the amino acid sequences of SEQ ID NOs: 34 and 62;
e) an HC comprising the amino acid sequences of SEQ ID NOs: 43 and 61 and an LC comprising the amino acid sequences of SEQ ID NOs: 44 and 62; or
f) an HC comprising the amino acid sequences of SEQ ID NOs: 53 and 61 and an LC comprising the amino acid sequences of SEQ ID NOs: 54 and 62.

In some embodiments, the anti-CD40 antibody or antigen-binding portion has a heavy chain CDR3 (H-CDR3) amino acid sequence of SEQ ID NO: 7, 17, 27, 37, 47, or 57.

In some embodiments, the anti-CD40 antibody or antigen-binding portion has heavy chain CDR1-3 (H-CDR1-3) comprising the amino acid sequences of SEQ ID NOs: 5-7, 15-17, 25-27, 35-37, 45-47, or 55-57, respectively.

In some embodiments, the anti-CD40 antibody or antigen-binding portion has a heavy chain variable domain (VH) amino acid sequence that is at least 80%, 85%, 90%, 91%, 92%, 93%, 94%, 95%, 96%, 97%, 98%, or 99% identical to the amino acid sequence of SEQ ID NO: 3, 13, 23, 33, 43, or 53.

In some embodiments, the anti-CD40 antibody or antigen-binding portion has a VH comprising the amino acid sequence of SEQ ID NO: 3, 13, 23, 33, 43, or 53.

In some embodiments, the anti-CD40 antibody has a VH amino acid sequence that is at least 80%, 85%, 90%, 91%, 92%, 93%, 94%, 95%, 96%, 97%, 98%, or 99% identical to the amino acid sequence of SEQ ID NO: 3, 13, 23, 33, 43, or 53; and a heavy chain constant region amino acid sequence that is at least 80%, 85%, 90%, 91%, 92%, 93%, 94%, 95%, 96%, 97%, 98%, or 99% identical to the amino acid sequence of SEQ ID NO: 61.

In some embodiments, the anti-CD40 antibody comprises a VH amino acid sequence of SEQ ID NO: 3, 13, 23, 33, 43, or 53 and a heavy chain constant region amino acid sequence of SEQ ID NO: 61.

In some embodiments, the anti-CD40 antibody or antigen-binding portion has a light chain CDR3 (L-CDR3) amino acid sequence of SEQ ID NO: 10, 20, 30, 40, 50, or 60.

In some embodiments, the anti-CD40 antibody or antigen-binding portion has light chain CDR1-3 (L-CDR1-3) comprising the amino acid sequences of SEQ ID NOs: 8-10, 18-20, 28-30, 38-40, 48-50, or 58-60, respectively.

In some embodiments, the anti-CD40 antibody or antigen-binding portion has a light chain variable domain (VL) amino acid sequence that is at least 80%, 85%, 90%, 91%, 92%, 93%, 94%, 95%, 96%, 97%, 98%, or 99% identical to the amino acid sequence of SEQ ID NO: 4, 14, 24, 34, 44, or 54.

In some embodiments, the anti-CD40 antibody or antigen-binding portion has a VL comprising the amino acid sequence of SEQ ID NO: 4, 14, 24, 34, 44, or 54.

In some embodiments, the anti-CD40 antibody has a VL amino acid sequence that is at least 80%, 85%, 90%, 91%, 92%, 93%, 94%, 95%, 96%, 97%, 98%, or 99% identical to the amino acid sequence of SEQ ID NO: 4, 14, 24, 34, 44, or 54; and a light chain constant region amino acid sequence that is at least 80%, 85%, 90%, 91%, 92%, 93%, 94%, 95%, 96%, 97%, 98%, or 99% identical to the amino acid sequence of SEQ ID NO: 62.

In some embodiments, the anti-CD40 antibody comprises a VL amino acid sequence of SEQ ID NO: 4, 14, 24, 34, 44, or 54 and a light chain constant region amino acid sequence of SEQ ID NO: 62.

In certain embodiments, the anti-CD40 antibody comprises any one of the above-described heavy chains and any one of the above-described light chains.

In some embodiments, the anti-CD40 antibody or antigen-binding portion of the present disclosure comprises the H-CDR1-3 and L-CDR1-3 amino acid sequences of:
a) SEQ ID NOs: 5-10, respectively;
b) SEQ ID NOs: 15-20, respectively;
c) SEQ ID NOs: 25-30, respectively;
d) SEQ ID NOs: 35-40, respectively;
e) SEQ ID NOs: 45-50, respectively; or
f) SEQ ID NOs: 55-60, respectively.

In some embodiments, the anti-CD40 antibody or antigen-binding portion of the present disclosure comprises a VH and a VL that are 80%, 85%, 90%, 91%, 92%, 93%, 94%, 95%, 96%, 97%, 98%, or 99% identical to the amino acid sequences of:
a) SEQ ID NOs: 3 and 4, respectively;
b) SEQ ID NOs: 13 and 14, respectively;
c) SEQ ID NOs: 23 and 24, respectively;
d) SEQ ID NOs: 33 and 34, respectively;
e) SEQ ID NOs: 43 and 44, respectively; or
f) SEQ ID NOs: 53 and 54, respectively.

In some embodiments, the anti-CD40 antibody or antigen-binding portion of the present disclosure comprises a VH and a VL that comprise the amino acid sequences of:
a) SEQ ID NOs: 3 and 4, respectively;
b) SEQ ID NOs: 13 and 14, respectively;
c) SEQ ID NOs: 23 and 24, respectively;
d) SEQ ID NOs: 33 and 34, respectively;
e) SEQ ID NOs: 43 and 44, respectively; or
f) SEQ ID NOs: 53 and 54, respectively.

In some embodiments, the anti-CD40 antibody of the present disclosure comprises:
a) an HC comprising the amino acid sequences of SEQ ID NOs: 3 and 61 and an LC comprising the amino acid sequences of SEQ ID NOs: 4 and 62;
b) an HC comprising the amino acid sequences of SEQ ID NOs: 13 and 61 and an LC comprising the amino acid sequences of SEQ ID NOs: 14 and 62;
c) an HC comprising the amino acid sequences of SEQ ID NOs: 23 and 61 and an LC comprising the amino acid sequences of SEQ ID NOs: 24 and 62;
d) an HC comprising the amino acid sequences of SEQ ID NOs: 33 and 61 and an LC comprising the amino acid sequences of SEQ ID NOs: 34 and 62;
e) an HC comprising the amino acid sequences of SEQ ID NOs: 43 and 61 and an LC comprising the amino acid sequences of SEQ ID NOs: 44 and 62; or
f) an HC comprising the amino acid sequences of SEQ ID NOs: 53 and 61 and an LC comprising the amino acid sequences of SEQ ID NOs: 54 and 62.

The present disclosure also provides an anti-CD40 antibody or an antigen-binding portion thereof that competes or cross-competes for binding with, or binds to the same epitope as, antibody 17303, 16040, 15833, 16154, 15888, or 15948.

In some embodiments, the anti-CD40 antibody or antigen-binding portion of the present disclosure comprises the H-CDR1-3 and L-CDR1-3 amino acid sequences of antibody 17303, 16040, 15833, 16154, 15888, or 15948.

In some embodiments, the anti-CD40 antibody or antigen-binding portion of the present disclosure comprises a VH and a VL that are at least 80%, 85%, 90%, 91%, 92%, 93%, 94%, 95%, 96%, 97%, 98%, or 99% identical in amino acid sequence to the VH and VL, respectively, of antibody 17303, 16040, 15833, 16154, 15888, or 15948.

In some embodiments, the anti-CD40 antibody or antigen-binding portion of the present disclosure comprises a VH and a VL that are the VH and VL, respectively, of antibody 17303, 16040, 15833, 16154, 15888, or 15948.

In some embodiments, the anti-CD40 antibody of the present disclosure is antibody 17303, 16040, 15833, 16154, 15888, or 15948, or an antibody with the same amino acid sequences as said antibody.

The class of an anti-CD40 antibody obtained by the methods described herein may be changed or switched with another class or subclass. In some embodiments of the present disclosure, a nucleic acid molecule encoding VL or VH is isolated using methods well known in the art such that it does not include nucleic acid sequences encoding CL or CH, respectively. The nucleic acid molecules encoding VL or VH then are operatively linked to a nucleic acid sequence encoding a CL or CH, respectively, from a different class of immunoglobulin molecule. This may be achieved using a vector or nucleic acid molecule that comprises a CL or CH sequence, as described above. For example, an anti-CD40 antibody that was originally IgM may be class switched to IgG. Further, the class switching may be used to convert one IgG subclass to another, e.g., from IgG₁ to IgG₂. A κ light chain constant region can be changed, e.g., to a λ light chain constant region, or vice-versa. An exemplary method for producing an antibody of the present disclosure with a desired Ig isotype comprises the steps of isolating a nucleic acid molecule encoding the heavy chain of an anti-CD40 antibody and a nucleic acid molecule encoding the light chain of an anti-CD40 antibody, obtaining the variable domain of the heavy chain, ligating a coding sequence for the variable domain of the heavy chain with a coding sequence for the constant region of a heavy chain of the desired isotype, expressing the light chain and the heavy chain encoded by the ligated sequence in a cell, and collecting the anti-CD40 antibody with the desired isotype.

An anti-CD40 antibody described herein may be an IgG, an IgM, an IgE, an IgA, or an IgD molecule, but is typically of the IgG isotype, e.g., of IgG subclass Ig IgG₂ₐ or IgG_{2b}, IgG₃ or IgG₄. In some embodiments, the antibody is of the isotype subclass IgG₁.

In some embodiments, an anti-CD40 antibody described herein may comprise at least one mutation in the Fc region. A number of different Fc mutations are known, where these mutations alter the antibody's effector function. For example, in some embodiments, the anti-CD40 antibody comprises at least one mutation in the Fc region that reduces effector function, e.g., mutations at one or more of positions 228, 233, 234 and 235, where amino acid positions are numbered according to the IMGT® numbering scheme.

In some embodiments, e.g., where the antibody is of the IgG₁ subclass, one or both of the amino acid residues at positions 234 and 235 may be mutated, for example from Leu to Ala (L234A/L235A). These mutations reduce effector function of the Fc region of IgG₁ antibodies. The amino acid positions are numbered according to the IMGT® numbering scheme.

In some embodiments, e.g., where the antibody is of the IgG₄ subclass, it may comprise the mutation S228P, where the amino acid position is numbered according to the IMGT® numbering scheme. This mutation is known to reduce undesired Fab arm exchange.

In some embodiments, an anti-CD40 antibody or antigen-binding portion described herein binds to human CD40 with a K_{D} of 2×10⁻⁸, 1×10⁻⁸, 9×10⁻⁹, 8×10⁻⁹, 7×10⁻⁹, 6×10⁻⁹, 5×10⁻⁹, 4×10⁻⁹, 3×10⁻⁹, 2×10⁻⁹, 1×10⁻⁹, 9×10⁻¹⁰, 8×10⁻¹⁰, 7×10⁻¹⁰, 6×10⁻¹⁰, or 5×10⁻¹⁰ M or less.

In some embodiments, an anti-CD40 antibody or antigen-binding portion described herein binds to cynomolgus CD40 with a K_{D} of 6×10⁻⁸, 5×10⁻⁸, 4×10⁻⁸, 3×10⁻⁸, 2×10⁻⁸×10⁻⁸, 1×10⁻⁸, 9×10⁻⁹, 8×10⁻⁹, 7×10⁻⁹, 6×10⁻⁹, 5×10⁻⁹, 4×10⁻⁹, 3×10⁻⁹, 2×10⁻⁹, 1×10⁻⁹, 9×10⁻¹⁰, 8×10⁻¹⁰, 7×10⁻¹⁰, 6×10⁻¹⁰, 5×10⁻¹⁰, 4×10⁻¹⁰, or 3×10⁻¹⁰ M or less.

In some embodiments, an anti-CD40 antibody or antigen-binding portion described herein stimulates CD40 activity, e.g., at a concentration of about 1, 5, 10, 15, 20, or 25 μg/mL or less, in a CD40-NFkB-luciferase reporter assay (e.g., as described in the Examples below).

In some embodiments, an anti-CD40 antibody or antigen-binding portion described herein does not inhibit CD40 ligand activity, e.g., at a concentration of up to 10 μg/mL, in a CD40-NFkB-luciferase reporter assay (e.g., as described in the Examples below).

In some embodiments, an anti-CD40 antibody or antigen-binding portion described herein stimulates proliferation of B cells in vitro, e.g., at a concentration of about 1, 5, 10, 15, 20, or 25 μg/mL or less, in the presence of IL-21 (e.g., at 50 ng/mL).

The present disclosure also contemplates an anti-CD40 antibody or antigen-binding portion described herein with any combination of the above properties.

In some embodiments, an anti-CD40 antibody or antigen-binding portion described herein has at least one (e.g., 1, 2, 3, 4, or all 5) of the following properties:
a) binds to human CD40 with a K_{D} of 8×10⁻⁹ M or less;
b) binds to cynomolgus CD40 with a K_{D} of 6×10⁻⁸ M or less;
c) stimulates CD40 activity in a CD40-NFkB-luciferase reporter assay (e.g., at a concentration of 25 μg/mL or less);
d) does not inhibit CD40 ligand activity in a CD40-NFkB-luciferase reporter assay (e.g., at a concentration of up to 10 μg/mL), and
e) stimulates proliferation of B cells in vitro in the presence of IL-21 (e.g., wherein the antibody or antigen-binding portion is at a concentration of 25 μg/mL or less, and the IL-21 is at a concentration of 50 ng/mL).

In some embodiments, an anti-CD40 antibody or antigen-binding portion described herein may inhibit tumor growth and/or induce tumor growth regression in vivo. In some embodiments, an anti-CD40 antibody or antigen-binding portion described herein may prolong survival of a cancer patient. Any combination of the above properties is also contemplated.

In certain embodiments, an antibody or antigen-binding portion thereof of the present disclosure may be part of a larger immunoadhesin molecule, formed by covalent or noncovalent association of the antibody or antibody portion with one or more other proteins or peptides. Examples of such immunoadhesin molecules include use of the streptavidin core region to make a tetrameric scFv molecule (Kipriyanov et al., Human Antibodies and Hybridomas 6:93-101 (1995)) and use of a cysteine residue, a marker peptide and a C-terminal polyhistidine tag to make bivalent and biotinylated scFv molecules (Kipriyanov et al., Mol. Immunol. 31:1047-1058 (1994)). Other examples include where one or more CDRs from an antibody are incorporated into a molecule either covalently or noncovalently to make it an immunoadhesin that specifically binds to an antigen of interest. In such embodiments, the CDR(s) may be incorporated as part of a larger polypeptide chain, may be covalently linked to another polypeptide chain, or may be incorporated noncovalently.

In some embodiments, a fusion antibody or immunoadhesin may be made that comprises all or a portion of an anti-CD40 antibody of the present disclosure linked to another polypeptide. In certain embodiments, only the variable domains of the anti-CD40 antibody are linked to the polypeptide. In certain embodiments, the VH domain of an anti-CD40 antibody is linked to a first polypeptide, while the VL domain of an anti-CD40 antibody is linked to a second polypeptide that associates with the first polypeptide in a manner such that the VH and VL domains can interact with one another to form an antigen-binding site. In some embodiments, the VH domain is separated from the VL domain by a linker such that the VH and VL domains can interact with one another (e.g., single-chain antibodies). The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bi-specific antibody.

To create a single chain antibody (scFv), the VH- and VL-encoding DNA fragments are operatively linked to another fragment encoding a flexible linker, e.g., encoding the amino acid sequence (Gly₄-Ser)₃ (SEQ ID NO: 64), such that the VH and VL sequences can be expressed as a contiguous single-chain protein, with the VL and VH domains joined by the flexible linker. See, e.g., Bird et al., Science 242:423-426 (1988); Huston et al., Proc. Natl. Acad. Sci. USA 85:5879-5883 (1988); and McCafferty et al., Nature 348:552-554 (1990). The single chain antibody may be monovalent, if only a single VH and VL are used; bivalent, if two VH and VL are used; or polyvalent, if more than two VH and VL are used. Bi-specific or polyvalent antibodies may be generated that bind specifically to human CD40 and to another molecule, for instance.

In other embodiments, other modified antibodies may be prepared using anti-CD40 antibody-encoding nucleic acid molecules. For instance, “kappa bodies” (III et al., Protein Eng. 10:949-57 (1997)), “minibodies” (Martin et al., EMBO J. 13:5303-9 (1994)), “diabodies” (Holliger et al., Proc. Natl. Acad. Sci. USA 90:6444-6448 (1993)), or “Janusins” (Traunecker et al., EMBO J. 10:3655-3659 (1991) and Traunecker et al., Int. J. Cancer (Suppl.) 7:51-52 (1992)) may be prepared using standard molecular biological techniques following the teachings of the specification.

An anti-CD40 antibody or antigen-binding portion of the present disclosure can be derivatized or linked to another molecule (e.g., another peptide or protein). In general, the antibodies or portions thereof are derivatized such that CD40 binding is not affected adversely by the derivatization or labeling. Accordingly, the antibodies and antibody portions of the present disclosure are intended to include both intact and modified forms of the human anti-CD40 antibodies described herein. For example, an antibody or antibody portion of the present disclosure can be functionally linked (by chemical coupling, genetic fusion, noncovalent association or otherwise) to one or more other molecular entities, such as another antibody (e.g., a bi-specific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bi-specific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available, e.g., from Pierce Chemical Company, Rockford, Ill.

An anti-CD40 antibody or antigen-binding portion can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody or antigen-binding portion according to the present disclosure may also be labeled. As used herein, the terms “label” or “labeled” refer to incorporation of another molecule in the antibody. In some embodiments, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In some embodiments, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In certain embodiments, the antibodies of the present disclosure may be present in a neutral form (including zwitterionic forms) or as a positively or negatively-charged species. In some embodiments, the antibodies may be complexed with a counterion to form a pharmaceutically acceptable salt.

### Anti-CD40 Antibody Compositions

The present disclosure also provides a combination therapy (e.g., a composition) that comprises one, two, three, four, or more of the anti-CD40 antibodies or antigen-binding portions thereof described herein. In certain embodiments, the combination therapy (e.g., composition) comprises two of the anti-CD40 antibodies or antigen-binding portions. The combination therapy may take the form of, e.g., a method of treatment using said antibodies or antigen-binding portions or a pharmaceutical composition comprising said antibodies or antigen-binding portions.

In some embodiments, the present disclosure provides a composition comprising a first anti-CD40 antibody or an antigen-binding portion thereof and a second anti-CD40 antibody or an antigen-binding portion thereof, wherein the first and second antibodies are:
antibodies 17303 and 16040, respectively;
antibodies 17303 and 15833, respectively;
antibodies 17303 and 16154, respectively;
antibodies 17303 and 15888, respectively;
antibodies 17303 and 15948, respectively;
antibodies 16040 and 15833, respectively;
antibodies 16040 and 16154, respectively;
antibodies 16040 and 15888, respectively;
antibodies 16040 and 15948, respectively;
antibodies 15833 and 16154, respectively;
antibodies 15833 and 15888, respectively;
antibodies 15833 and 15948, respectively;
antibodies 16154 and 15888, respectively;
antibodies 16154 and 15948, respectively; or
antibodies 15888 and 15948, respectively.

In some embodiments, the composition comprises antibodies or antigen-binding portions thereof that bind to the same epitope as, or compete for binding with, said first and second antibodies.

In some embodiments, the composition comprises an antibody or an antigen-binding portion thereof that comprises the H-CDR1-3 and L-CDR1-3 amino acid sequences of said first antibody, and an antibody or an antigen-binding portion thereof that comprises the H-CDR1-3 and L-CDR1-3 amino acid sequences of said second antibody.

In some embodiments, the composition comprises an antibody or an antigen-binding portion thereof that comprises a VH and a VL with amino acid sequences that are at least 80%, 85%, 90%, 91%, 92%, 93%, 94%, 95%, 96%, 97%, 98%, or 99% identical to the VH and VL amino acid sequences, respectively, of said first antibody, and an antibody or an antigen-binding portion thereof that comprises a VH and a VL with amino acid sequences that are at least 80%, 85%, 90%, 91%, 92%, 93%, 94%, 95%, 96%, 97%, 98%, or 99% identical to the VH and VL amino acid sequences, respectively, of said second antibody.

In some embodiments, the composition comprises an antibody or an antigen-binding portion thereof that comprises the VH and VL amino acid sequences of said first antibody, and an antibody or an antigen-binding portion thereof that comprises the VH and VL amino acid sequences of said second antibody.

In some embodiments, the composition comprises an antibody or an antigen-binding portion thereof that comprises the HC and LC amino acid sequences of said first antibody, and an antibody or an antigen-binding portion thereof that comprises the HC and LC amino acid sequences of said second antibody.

In certain embodiments, said composition may comprise one, two, or more antibodies or antigen-binding portions thereof selected from the group consisting of:
a) an antibody comprising H-CDR1-3 that comprise the amino acid sequences of SEQ ID NOs: 5-7, 15-17, 25-27, 35-37, 45-47, or 55-57, respectively;
b) an antibody whose VH is at least 90% identical in sequence to the amino acid sequence of SEQ ID NO: 3, 13, 23, 33, 43, or 53;
c) an antibody whose VH comprises the amino acid sequence of SEQ ID NO: 3, 13, 23, 33, 43, or 53;
d) an antibody whose HC comprises the amino acid sequences of SEQ ID NOs: 3 and 61, 13 and 61, 23 and 61, 33 and 61, 43 and 61, or 53 and 61;
e) an antibody comprising L-CDR1-3 that comprise the amino acid sequences of SEQ ID NOs: 8-10, 18-20, 28-30, 38-40, 48-50, or 58-60, respectively;
f) an antibody whose VL is at least 90% identical in sequence to the amino acid sequence of SEQ ID NO: 4, 14, 24, 34, 44, or 54;
g) an antibody whose VL comprises the amino acid sequence of SEQ ID NO: 4, 14, 24, 34, 44, or 54;
h) an antibody whose LC comprises the amino acid sequences of SEQ ID NOs: 4 and 62, 14 and 62, 24 and 62, 34 and 62, 44 and 62, or 54 and 62;
i) an antibody whose H-CDR1-3 and L-CDR1-3 comprise the amino acid sequences of SEQ ID NOs: 5-10, 15-20, 25-30, 35-40, 45-50, or 55-60, respectively;
j) an antibody comprising VH and VL that comprise amino acid sequences at least 90% identical to the amino acid sequences of SEQ ID NOs: 3 and 4, 13 and 14, 23 and 24, 33 and 34, 43 and 44, or 53 and 54, respectively;
k) an antibody comprising VH and VL that comprise the amino acid sequences of SEQ ID NOs: 3 and 4, 13 and 14, 23 and 24, 33 and 34, 43 and 44, or 53 and 54, respectively; and
l) an antibody comprising HC and LC that comprise the amino acid sequences of 3 and 61, and 4 and 62; 13 and 61, and 14 and 62; 23 and 61, and 24 and 62; 33 and 61, and 34 and 62; 43 and 61, and 44 and 62; or 53 and 61, and 54 and 62; respectively.

In some embodiments, the present disclosure provides a composition comprising:
a first antibody or antigen-binding portion thereof that comprises H-CDR1-3 and L-CDR1-3 amino acid sequences of SEQ ID NOs: 5-10, respectively; and a second antibody or antigen-binding portion thereof that comprises H-CDR1-3 and L-CDR1-3 amino acid sequences of SEQ ID NOs: 15-20, respectively;
a first antibody or antigen-binding portion thereof that comprises H-CDR1-3 and L-CDR1-3 amino acid sequences of SEQ ID NOs: 5-10, respectively; and a second antibody or antigen-binding portion thereof that comprises H-CDR1-3 and L-CDR1-3 amino acid sequences of SEQ ID NOs: 25-30, respectively;
a first antibody or antigen-binding portion thereof that comprises H-CDR1-3 and L-CDR1-3 amino acid sequences of SEQ ID NOs: 5-10, respectively; and a second antibody or antigen-binding portion thereof that comprises H-CDR1-3 and L-CDR1-3 amino acid sequences of SEQ ID NOs: 55-60, respectively;
a first antibody or antigen-binding portion thereof that comprises H-CDR1-3 and L-CDR1-3 amino acid sequences of SEQ ID NOs: 15-20, respectively; and a second antibody or antigen-binding portion thereof that comprises H-CDR1-3 and L-CDR1-3 amino acid sequences of SEQ ID NOs: 45-50, respectively; or
a first antibody or antigen-binding portion thereof that comprises H-CDR1-3 and L-CDR1-3 amino acid sequences of SEQ ID NOs: 15-20, respectively; and a second antibody or antigen-binding portion thereof that comprises H-CDR1-3 and L-CDR1-3 amino acid sequences of SEQ ID NOs: 35-40, respectively.

In some embodiments, the present disclosure provides a composition comprising:
a first antibody or antigen-binding portion thereof that comprises VH and VL amino acid sequences of SEQ ID NOs: 3 and 4, respectively; and a second antibody or antigen-binding portion thereof that comprises VH and VL amino acid sequences of SEQ ID NOs: 13 and 14, respectively;
a first antibody or antigen-binding portion thereof that comprises VH and VL amino acid sequences of SEQ ID NOs: 3 and 4, respectively; and a second antibody or antigen-binding portion thereof that comprises VH and VL amino acid sequences of SEQ ID NOs: 23 and 24, respectively;
a first antibody or antigen-binding portion thereof that comprises VH and VL amino acid sequences of SEQ ID NOs: 3 and 4, respectively; and a second antibody or antigen-binding portion thereof that comprises VH and VL amino acid sequences of SEQ ID NOs: 53 and 54, respectively;
a first antibody or antigen-binding portion thereof that comprises VH and VL amino acid sequences of SEQ ID NOs: 13 and 14, respectively; and a second antibody or antigen-binding portion thereof that comprises VH and VL amino acid sequences of SEQ ID NOs: 43 and 44, respectively; or
a first antibody or antigen-binding portion thereof that comprises VH and VL amino acid sequences of SEQ ID NOs: 13 and 14, respectively; and a second antibody or antigen-binding portion thereof that comprises VH and VL amino acid sequences of SEQ ID NOs: 33 and 34, respectively.

In some embodiments, the present disclosure provides a composition comprising:
a first antibody or antigen-binding portion thereof that comprises an HC comprising the amino acid sequences of SEQ ID NOs: 3 and 61 and an LC comprising the amino acid sequences of SEQ ID NOs: 4 and 62; and a second antibody or antigen-binding portion thereof that comprises an HC comprising the amino acid sequences of SEQ ID NOs: 13 and 61 and an LC comprising the amino acid sequences of SEQ ID NOs: 14 and 62;
a first antibody or antigen-binding portion thereof that comprises an HC comprising the amino acid sequences of SEQ ID NOs: 3 and 61 and an LC comprising the amino acid sequences of SEQ ID NOs: 4 and 62; and a second antibody or antigen-binding portion thereof that comprises an HC comprising the amino acid sequences of SEQ ID NOs: 23 and 61 and an LC comprising the amino acid sequences of SEQ ID NOs: 24 and 62;
a first antibody or antigen-binding portion thereof that comprises an HC comprising the amino acid sequences of SEQ ID NOs: 3 and 61 and an LC comprising the amino acid sequences of SEQ ID NOs: 4 and 62; and a second antibody or antigen-binding portion thereof that comprises an HC comprising the amino acid sequences of SEQ ID NOs: 53 and 61 and an LC comprising the amino acid sequences of SEQ ID NOs: 54 and 62;
a first antibody or antigen-binding portion thereof that comprises an HC comprising the amino acid sequences of SEQ ID NOs: 13 and 61 and an LC comprising the amino acid sequences of SEQ ID NOs: 14 and 62; and a second antibody or antigen-binding portion thereof that comprises an HC comprising the amino acid sequences of SEQ ID NOs: 43 and 61 and an LC comprising the amino acid sequences of SEQ ID NOs: 44 and 62; or
a first antibody or antigen-binding portion thereof that comprises an HC comprising the amino acid sequences of SEQ ID NOs: 13 and 61 and an LC comprising the amino acid sequences of SEQ ID NOs: 14 and 62; and a second antibody or antigen-binding portion thereof that comprises an HC comprising the amino acid sequences of SEQ ID NOs: 33 and 61 and an LC comprising the amino acid sequences of SEQ ID NOs: 34 and 62.

In some embodiments, an anti-CD40 antibody of the invention, or an antigen-binding portion thereof, binds to an epitope of CD40 that includes at least one (e.g., at least 1, 2, 3, 4, 5, 6, 7, 8, 9, 10, 11, 12, 13, 14, 15, 16, 17, 18, 19, or 20) of the following residues of SEQ ID NO: 63: R27, E28, K29, Q30, 133, N34, S49, E53, F54, T55, E58, H110, E114, H122, R123, C125, G128, F129, G130, V131, K132, Q133, 1134, A135, T136, G137, V138, D140, and 1142. In certain embodiments, the antibody or antigen-binding portion binds to an epitope of CD40 that comprises or consists of residues N34, S49, E53-T55, and E58 (such as antibody 17303). In certain embodiments, the antibody or antigen-binding portion binds to an epitope of CD40 that comprises or consists of residues H110, H122, C125, G128-V131, Q133-V138, D140, and 1142 (such as antibody 16040). In certain embodiments, the antibody or antigen-binding portion binds to an epitope of CD40 that comprises or consists of residues H110, E114, R123, C125, G128-Q133, A135, T136, V138, D140, and 1142 (such as antibody 15833).

In some embodiments, an anti-CD40 antibody of the invention, or an antigen-binding portion thereof, binds to an epitope of CD40 that comprises residues 26-45, 31-65, 106-115, 106-145, 131-145, or any combination thereof, of SEQ ID NO: 63. In certain embodiments, the antibody or antigen-binding portion binds to an epitope of CD40 that comprises or consists of residues 31-65 (such as antibody 17303). In certain embodiments, the antibody or antigen-binding portion binds to an epitope of CD40 that comprises or consists of residues 106-115 and 131-145 (such as antibody 16040). In certain embodiments, the antibody or antigen-binding portion binds to an epitope of CD40 that comprises or consists of residues 106-145 (such as antibody 15833).

An epitope with any combination of the above residues, or the residues shown in Table 8, is also contemplated.

In some embodiments, an amino acid sequence comprising a CD40 epitope as described herein can be used as an immunogen (e.g., administered to an animal or as an antigen for screening antibody libraries) to generate or identify anti-CD40 antibodies or antigen-binding portions thereof that bind to said epitope.

In some embodiments, the anti-CD40 antibody composition stimulates CD40 activity, e.g., at a concentration of about 1, 5, 10, 15, 20, or 25 μg/mL or less in a CD40-NFkB-luciferase reporter assay (e.g., as described in the Examples below).

In some embodiments, the anti-CD40 antibody composition does not inhibit CD40 ligand activity, e.g., at a concentration of up to 10 μg/mL in a CD40-NFkB-luciferase reporter assay (e.g., as described in the Examples below).

In some embodiments, the anti-CD40 antibody composition stimulates CD40 activity, e.g., at a concentration of about 1, 5, 10, 15, 20, or 25 μg/mL or less in a one-way MLR assay (e.g., as described in the Examples below). CD40 activity may be measured, e.g., as an increase in IFN-γ levels. In certain embodiments, at a concentration of 25 μg/mL, the anti-CD40 antibody composition increases IFN-γ levels to at least about 125, 150, 175, 200, 225, 250, 275, or 300%.

In some embodiments, the anti-CD40 antibody composition stimulates proliferation of B cells in vitro, e.g., at a concentration of about 1, 5, 10, 15, 20, or 25 μg/mL or less in the presence of IL-21 (e.g., at 50 ng/mL).

In some embodiments, the anti-CD40 antibody composition stimulates dendritic cell activation in vitro, e.g., at a concentration of about 1, 5, 10, 15, 20, or 25 μg/mL or less. Dendritic cell activation may be measured, e.g., as an increase in IL-12p40 secretion. In certain embodiments, e.g., at a concentration of 25 μg/mL, the anti-CD40 antibody composition increases IL-12p40 secretion to at least about 125, 150, 175, 200, 225, 250, 275, or 300%.

In some embodiments, the anti-CD40 antibody composition stimulates proliferation of B cells from splenocytes (e.g., as described in the Examples below), e.g., at a concentration of about 100, 1000, or 10000 ng/mL or less. In certain embodiments, at a concentration of 10000 ng/mL, the anti-CD40 composition increases the survival rate of the B cells by at least about 100, 500, 1000, 1500, 2000, 2500, 3000, 3500, 4000, 4500, 5000, 5500, 6000, 6500, 7000, 7500, or 8000%. In particular embodiments, the anti-CD40 composition is more potent than selicrelumab, CD40L, or both, in stimulating the proliferation of B cells from splenocytes.

In some embodiments, the anti-CD40 antibody composition inhibits tumor growth in vivo in huCD34-NOG mice engrafted with Raji cells (e.g., as described in the Examples below), for example at a concentration of about 5 mg/kg. In certain embodiments, the anti-CD40 compositions inhibits tumor growth by at least about 10, 20, 30, 40, 50, 60, 70, 80, 90, or 100%.

In some embodiments, the anti-CD40 antibody composition inhibits tumor growth in vivo in CD40 HuGEMM mice engrafted with MC38-OVA colon carcinoma cells (e.g., as described in the Examples below), for example at a concentration of about 5, 10, 15, or 20 mg/kg. In certain embodiments, the anti-CD40 compositions inhibits tumor growth by at least about 10, 20, 30, 40, 50, 60, 70, 80, 90, or 100%.

In some embodiments, in vivo administration of the anti-CD40 antibody composition does not result in toxicity based on body weight measurements.

The present disclosure also contemplates an anti-CD40 antibody composition with any combination of the above properties.

In some embodiments, an anti-CD40 antibody composition described herein has at least one (e.g., 1, 2, 3, 4, 5, 6, 7, or all 8) of the following properties:
a) stimulates CD40 activity in a CD40-NFkB-luciferase reporter assay (e.g., at a concentration of 25 μg/mL or less);
b) does not inhibit CD40 ligand activity in a CD40-NFkB-luciferase reporter assay (e.g., at a concentration of up to 10 μg/mL);
c) stimulates CD40 activity in a one-way MLR assay (e.g., at a concentration of 25 μg/mL or less);
d) stimulates proliferation of B cells in vitro in the presence of IL-21 (e.g., wherein the composition is at a concentration of 25 μg/mL or less, and the IL-21 is at a concentration of 50 ng/mL);
e) stimulates dendritic cell activation in vitro (e.g., at a concentration of 25 μg/mL or less);
f) stimulates proliferation of B cells from splenocytes (e.g., at a concentration of 100, 1000, or 10000 ng/mL);
g) inhibits tumor growth in vivo in huCD34-NOG mice engrafted with Raji cells (e.g., at a concentration of 5 mg/kg); and
h) inhibits tumor growth in vivo in CD40 HuGEMM mice engrafted with MC38-OVA colon carcinoma cells (e.g., at a concentration of 20 mg/kg).

In some embodiments, an anti-CD40 antibody composition described herein may inhibit tumor growth and/or induce tumor growth regression in vivo. In some embodiments, an anti-CD40 antibody composition described herein may prolong survival of a cancer patient

The present disclosure also provides a method for producing an anti-CD40 antibody composition described herein, comprising providing a first anti-CD40 antibody or antigen-binding portion and a second anti-CD40 antibody or antigen-binding portion, and admixing the two antibodies or portions.

### Bi-Specific Binding Molecules

In a further aspect, the present disclosure provides a bi-specific binding molecule having the binding specificity (e.g., comprising the antigen-binding portions, such as the six CDRs or the VH and VL) of an anti-CD40 antibody described herein and the binding specificity of another, distinct anti-CD40 antibody (e.g., another anti-CD40 antibody described herein) or an antibody that targets a different protein, such as a cancer antigen or another cell surface molecule whose activity mediates a disease condition such as cancer. Such bi-specific binding molecules are known in the art, and examples of different types of bi-specific binding molecules are given elsewhere herein.

### Nucleic Acid Molecules and Vectors

The present disclosure also provides nucleic acid molecules and sequences encoding anti-CD40 antibodies or antigen-binding portions thereof described herein. In some embodiments, different nucleic acid molecules encode the heavy chain and light chain amino acid sequences of the anti-CD40 antibody or antigen-binding portion. In other embodiments, the same nucleic acid molecule encodes the heavy chain and light chain amino acid sequences of the anti-CD40 antibody or antigen-binding portion.

A reference to a nucleotide sequence encompasses its complement unless otherwise specified. Thus, a reference to a nucleic acid having a particular sequence should be understood to encompass its complementary strand, with its complementary sequence. The term “polynucleotide” as referred to herein means a polymeric form of nucleotides of at least 10 bases in length, either ribonucleotides or deoxynucleotides or a modified form of either type of nucleotide. The term includes single- and double-stranded forms.

In some embodiments, the present disclosure provides a nucleic acid molecule comprising a nucleotide sequence that encodes the heavy chain or an antigen-binding portion thereof, or a nucleotide sequence that encodes the light chain or an antigen-binding portion thereof, or both, of an anti-CD40 antibody or antigen-binding portion thereof described herein.

The present disclosure also provides nucleotide sequences that are at least 70%, 75%, 80%, 85%, 90%, 95%, 97%, 98% or 99% identical to one or more nucleotide sequences recited herein, e.g., to a nucleotide sequence selected from the group consisting of SEQ ID NOs: 1, 2, 11, 12, 21, 22, 31, 32, 41, 42, 51, and 52, or to a nucleotide sequence encoding an amino acid sequence selected from the group consisting of SEQ ID NOs: 3, 4, 13, 14, 23, 24, 33, 34, 43, 44, 53, and 54. The term “percent sequence identity” in the context of nucleic acid sequences refers to the residues in two sequences that are the same when aligned for maximum correspondence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (see, e.g., Pearson, Methods Enzymol. 183:63-98 (1990); Pearson, Methods Mol. Biol. 132:185-219 (2000); Pearson, Methods Enzymol. 266:227-258 (1996); and Pearson, J. Mol. Biol. 276:71-84 (1998); incorporated herein by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, incorporated herein by reference.

In some embodiments, the present disclosure provides a nucleic acid molecule comprising a nucleotide sequence selected from the group consisting of SEQ ID NOs: 1, 2, 11, 12, 21, 22, 31, 32, 41, 42, 51, and 52. In certain embodiments, the nucleic acid molecule comprises the nucleotide sequences of SEQ ID NOs: 1 and 2, 11 and 12, 21 and 22, 31 and 32, 41 and 42, or 51 and 52.

In any of the above embodiments, the nucleic acid molecules may be isolated. Nucleic acid molecules referred to herein as “isolated” or “purified” are nucleic acids which (1) have been separated away from the nucleic acids of the genomic DNA or cellular RNA of their source of origin; and/or (2) do not occur in nature.

In a further aspect, the present disclosure provides a vector suitable for expressing one or both of the chains of an antibody or antigen-binding portion thereof as described herein. The term “vector”, as used herein, means a nucleic acid molecule capable of transporting another nucleic acid to which it has been linked. In some embodiments, the vector is a plasmid, i.e., a circular double stranded piece of DNA into which additional DNA segments may be ligated. Moreover, certain vectors are capable of directing the expression of genes to which they are operatively linked. Such vectors are referred to herein as “recombinant expression vectors” (or simply, “expression vectors”).

The present disclosure provides vectors comprising nucleic acid molecules that encode the heavy chain, the light chain, or both the heavy and light chains of an anti-CD40 antibody as described herein or an antigen-binding portion thereof. In certain embodiments, a vector of the present disclosure comprises a nucleic acid molecule described herein. The present disclosure further provides vectors comprising nucleic acid molecules encoding fusion proteins, modified antibodies, antibody fragments, and probes thereof. The vector may further comprise an expression control sequence.

The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

In some embodiments, a nucleic acid molecule as described herein comprises a nucleotide sequence encoding a VH domain from an anti-CD40 antibody or antigen-binding portion as described herein joined in-frame to a nucleotide sequence encoding a heavy chain constant region from any source. Similarly, a nucleic acid molecule as described herein can comprise a nucleotide sequence encoding a VL domain from an anti-CD40 antibody or antigen-binding portion as described herein joined in-frame to a nucleotide sequence encoding a light chain constant region from any source.

In some embodiments of the present disclosure, nucleic acid molecules encoding the VH and/or VL may be “converted” to full-length antibody genes. In some embodiments, nucleic acid molecules encoding the VH or VL domains are converted to full-length antibody genes by insertion into an expression vector already encoding heavy chain constant (CH) or light chain constant (CL) regions, respectively, such that the VH segment is operatively linked to the CH segment(s) within the vector, and/or the VL segment is operatively linked to the CL segment within the vector. In some embodiments, nucleic acid molecules encoding the VH and/or VL domains are converted into full-length antibody genes by linking, e.g., ligating, a nucleic acid molecule encoding a VH and/or VL domain to a nucleic acid molecule encoding a CH and/or CL region using standard molecular biological techniques. Nucleic acid molecules encoding the full-length heavy and/or light chains may then be expressed from a cell into which they have been introduced and the anti-CD40 antibody isolated.

In some embodiments, the framework region(s) are mutated so that the resulting framework region(s) have the amino acid sequence of the corresponding germline gene. A mutation may be made in a framework region or constant region, e.g., to increase the half-life of the anti-CD40 antibody. See, e.g., PCT Publication WO 00/09560. A mutation in a framework region or constant region also can be made to alter the immunogenicity of the antibody, and/or to provide a site for covalent or non-covalent binding to another molecule. According to the present disclosure, an antibody may have mutations in any one or more of the CDRs or framework regions of the variable domain or in the constant region.

### Host Cells and Methods of Antibody and Antibody Composition Production

The present disclosure also provides methods for producing the antibody compositions and antibodies and antigen-binding portions thereof described herein. In some embodiments, the present disclosure relates to a method for producing an anti-CD40 antibody or antigen-binding portion as described herein, comprising providing a host cell (e.g., a recombinant host cell) comprising a nucleotide sequence that encodes the heavy chain or an antigen-binding portion thereof, and a nucleotide sequence that encodes the light chain or an antigen-binding portion thereof, of an anti-CD40 antibody or antigen-binding portion described herein; cultivating said host cell under conditions suitable for expression of the antibody or antigen-binding portion; and isolating the resulting antibody or antigen-binding portion. Antibodies or antigen-binding portions produced by such expression in such recombinant host cells are referred to herein as “recombinant” antibodies or antigen-binding portions. The present disclosure also provides progeny cells of such host cells, and antibodies or antigen-binding portions produced by same.

The term “recombinant host cell” (or simply “host cell”), as used herein, means a cell into which a recombinant expression vector has been introduced. By definition, a recombinant host cell does not occur in nature. The present disclosure provides host cells that may comprise, e.g., a vector as described herein. The present disclosure also provides host cells that comprise, e.g., a nucleotide sequence encoding the heavy chain or an antigen-binding portion thereof, a nucleotide sequence encoding the light chain or an antigen-binding portion thereof, or both, of an anti-CD40 antibody or antigen-binding portion thereof described herein. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

Nucleic acid molecules encoding anti-CD40 antibodies and antigen-binding portions thereof and vectors comprising these nucleic acid molecules can be used for transfection of a suitable mammalian, plant, bacterial or yeast host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

It is likely that antibodies expressed by different cell lines or in transgenic animals will have different glycosylation patterns from each other. However, all antibodies encoded by the nucleic acid molecules provided herein, or comprising the amino acid sequences provided herein are part of the present disclosure, regardless of the glycosylation state of the antibodies, and more generally, regardless of the presence or absence of post-translational modification(s).

### Pharmaceutical Compositions

Another aspect of the present disclosure is a pharmaceutical composition comprising as an active ingredient (or as the sole active ingredient) an anti-CD40 antibody or antigen-binding portion thereof, antibody composition, or bi-specific binding molecule as described herein. The pharmaceutical composition may additionally comprise a pharmaceutically acceptable excipient. In some embodiments, the pharmaceutical compositions described herein are intended for enhancing or stimulating the immune system of a patient (e.g., enhancing a T cell-mediated immune response of a patient). In some embodiments, the pharmaceutical compositions are intended for amelioration, prevention, and/or treatment of cancer, e.g., a cancer described herein. In certain embodiments, the cancer is in a tissue such as skin, lung, intestine, colon, ovary, brain, prostate, kidney, soft tissues, the hematopoietic system, head and neck, liver, bone, bladder, breast, stomach, uterus, cervix, and pancreas. In certain embodiments, the cancer is melanoma, head and neck cancer, esophageal cancer, non-small cell lung cancer, pancreatic cancer, urothelial carcinoma, epithelial cancer, sarcoma, non-Hodgkin's lymphoma, B cell lymphoma, diffuse large B cell lymphoma, follicular lymphoma, chronic lymphocytic leukemia, or multiple myeloma.

Pharmaceutical compositions of the present disclosure will comprise one or more anti-CD40 antibodies, antigen-binding portions, antibody compositions, or bi-specific binding molecules of the present disclosure, e.g., one or two anti-CD40 antibodies, antigen-binding portions, or bi-specific binding molecules. In some embodiments, the composition comprises a single anti-CD40 antibody of the present disclosure or an antigen-binding portion thereof. In other embodiments, the composition comprises two distinct anti-CD40 antibodies of the present disclosure or antigen-binding portions thereof.

In some embodiments, the pharmaceutical composition may comprise at least one anti-CD40 antibody or antigen-binding portion thereof of the present disclosure, e.g., one anti-CD40 antibody or portion, and one or more additional antibodies that target one or more relevant cell surface receptors, e.g., one or more cancer-relevant receptors.

Generally, the antibodies, antigen-binding portions, and bi-specific binding molecules of the present disclosure are suitable to be administered as a formulation in association with one or more pharmaceutically acceptable excipient(s), e.g., as described below.

The term “excipient” is used herein to describe any ingredient other than the compound(s) of the present disclosure. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions of the present disclosure and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP (good manufacturing practices) conditions.

A pharmaceutical composition of the present disclosure may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation.

### Therapeutic Uses of Antibodies and Compositions of the Present Disclosure

In some embodiments, the anti-CD40 antibodies and antigen-binding portions thereof, anti-CD40 antibody compositions, and bi-specific binding molecules of the present disclosure are used to enhance or activate the immune system in a patient (e.g., a mammal such as a human) in need thereof, e.g., by stimulating CD40 activity. In certain embodiments, the patient is immune-suppressed.

In some embodiments, the anti-CD40 antibodies and antigen-binding portions thereof, anti-CD40 antibody compositions, and bi-specific binding molecules of the present disclosure are used to treat an immune disorder in a patient (e.g., a mammal such as a human) in need thereof. In certain embodiments, the patient is immunocompromised.

In some embodiments, the anti-CD40 antibodies and antigen-binding portions thereof, anti-CD40 antibody compositions, and bi-specific binding molecules of the present disclosure are used to treat cancer in a patient (e.g., a mammal such as a human). In certain embodiments, a physician can boost the anti-cancer activity of a patient's own immune system by administering an anti-CD40 antibody or antigen-binding portion thereof, composition, or bi-specific binding molecule as described herein. For example, a physician can boost anti-tumor activity in a patient by administering an anti-CD40 antibody, antibody composition, or bi-specific binding molecule of the present disclosure, alone or in combination with other therapeutic agents (sequentially or concurrently).

In some embodiments, the anti-CD40 antibodies, antigen-binding portions, compositions, and bi-specific binding molecules of the present disclosure are for use in the treatment of cancer. The cancer may be in one or more tissues such as skin, lung, intestine, colon, ovary, brain, prostate, kidney, soft tissues, the hematopoietic system, head and neck, liver, bone, bladder, breast, stomach, uterus, cervix, and pancreas.

In some embodiments, cancers treated by the anti-CD40 antibodies, antigen-binding portions, compositions, and bi-specific binding molecules of the present disclosure may include, e.g., melanoma (e.g., advanced or metastatic melanoma), skin basal cell cancer, glioblastoma, glioma, gliosarcoma, astrocytoma, meningioma, neuroblastoma, adrenocortical cancer, head and neck squamous cell cancer, oral cancer, salivary gland cancer, nasopharyngeal cancer, breast cancer, lung cancer (e.g., non-small cell lung cancer (NSCLC), small cell lung cancer, and squamous cell lung cancer), esophageal cancer, gastroesophageal junction cancer, gastric cancer, gastrointestinal cancer, primary peritoneal cancer, liver cancer, hepatocellular carcinoma, biliary tract cancer, colon cancer, colorectal carcinoma, ovarian cancer, fallopian tube cancer, bladder cancer, upper urinary tract cancer, urothelial cancer, renal cell carcinoma, kidney cancer, genitourinary cancer, cervical cancer, prostate cancer, fibrosarcoma, liposarcoma, rhabdomyosarcoma, osteosarcoma, histiocytoma, pancreatic cancer, endometrial cancer, cancer of the appendix, advanced Merkel cell cancer, multiple myeloma, sarcomas, choriocarcinoma, erythroleukemia, acute lymphoblastic leukemia, acute monocytic leukemia, acute promyelocytic leukemia, acute myeloid leukemia, chronic myeloid leukemia, chronic lymphocytic leukemia, acute lymphoblastic leukemia, mast cell leukemia, small lymphocytic lymphoma, Burkitt's lymphoma, Hodgkin's lymphoma, non-Hodgkin's lymphoma, diffuse large B cell lymphoma, follicular lymphoma, monocytic lymphoma, HTLV-associated T cell leukemia/lymphoma, mesothelioma, and solid tumors. The cancer may be, e.g., at an early, intermediate, late, locally advanced, or metastatic stage, and may be relapsed or refractory to other therapeutics (e.g., other anti-CD40 therapeutics) or there may be no standard therapy available.

In some embodiments, cancers treated by the anti-CD40 antibodies, antigen-binding portions, compositions, and/or bi-specific binding molecules of the present disclosure may include, e.g., melanoma, head and neck cancer, esophageal cancer, gastroesophageal cancer, lung cancer (e.g., NSCLC), breast cancer, ovarian cancer, pancreatic cancer, urothelial carcinoma, renal cancer, colon cancer, prostate cancer, epithelial cancer, mesothelioma, sarcoma, lymphoma (e.g., non-Hodgkin's lymphoma, B cell lymphoma, diffuse large B cell lymphoma, follicular lymphoma, etc.), leukemia (e.g., chronic lymphocytic leukemia), and multiple myeloma.

In some embodiments, the anti-CD40 antibodies, antigen-binding portions, compositions, and/or bi-specific binding molecules of the present disclosure are administered with an immunostimulatory agent, a vaccine, a chemotherapeutic agent, an anti-neoplastic agent, an anti-angiogenic agent, a tyrosine kinase inhibitor, or radiation therapy.

In some embodiments, the antibody or antigen-binding portion, composition, or bi-specific binding molecule is for use in treating viral and/or parasitic infections, e.g., where the pathogens inhibit the host immune response. The pathogen may be, e.g., HIV, hepatitis (A, B, or C), human papilloma virus (HPV), lymphocytic choriomeningitis virus (LCMV), adenovirus, flavivirus, echovirus, rhinovirus, coxsackie virus, coronavirus, respiratory syncytial virus, mumps virus, rotavirus, measles virus, rubella virus, parvovirus, vaccinia virus, human T cell lymphotrophic virus (HTLV), human cytomegalovirus (HCMV), dengue virus, molluscum virus, poliovirus, rabies virus, John Cunningham (JC) virus, arboviral encephalitis virus, simian immunodeficiency virus (SIV), influenza, herpes, Giardia, malaria, Leishmania, Staphylococcus aureus, or Pseudomonas aeruginosa.

In some embodiments, the antibody or antigen-binding portion, composition, or bi-specific binding molecule may be used to treat a patient who is, or is at risk of being, immunocompromised (e.g., due to chemotherapeutic or radiation therapy).

“Treat,” “treating,” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

“Therapeutically effective amount” refers to the amount of the therapeutic agent being administered that will relieve to some extent one or more of the symptoms of the disorder being treated. A therapeutically effective amount of an anti-cancer therapeutic may, for example, result in delayed tumor growth, tumor shrinkage, increased survival, elimination of cancer cells, slowed or decreased disease progression, reversal of metastasis, or other clinical endpoints desired by healthcare professionals.

The anti-CD40 antibodies or antigen-binding portions thereof, antibody compositions, or bi-specific binding molecules described herein may be administered alone or in combination with one or more other drugs or antibodies (or as any combination thereof). The pharmaceutical compositions, methods and uses described herein thus also encompass embodiments of combinations (co-administration) with other active agents, as detailed below.

As used herein, the terms “co-administration,” “co-administered” and “in combination with,” referring to the anti-CD40 antibodies and antigen-binding portions thereof, antibody compositions, and bi-specific binding molecules of the present disclosure with one or more other therapeutic agents, is intended to mean, and does refer to and include the following:
a) simultaneous administration of such combination of antibody/antigen-binding portion/antibody composition/bi-specific binding molecule of the present disclosure and therapeutic agent(s) to a patient in need of treatment, when such components are formulated together into a single dosage form which releases said components at substantially the same time to said patient,
b) substantially simultaneous administration of such combination of antibody/antigen-binding portion/antibody composition/bi-specific binding molecule of the present disclosure and therapeutic agent(s) to a patient in need of treatment, when such components are formulated apart from each other into separate dosage forms which are taken at substantially the same time by said patient, whereupon said components are released at substantially the same time to said patient,
c) sequential administration of such combination of antibody/antigen-binding portion/antibody composition/bi-specific binding molecule of the present disclosure and therapeutic agent(s) to a patient in need of treatment, when such components are formulated apart from each other into separate dosage forms which are taken at consecutive times by said patient with a significant time interval between each administration, whereupon said components are released at substantially different times to said patient; and
d) sequential administration of such combination of antibody/antigen-binding portion/antibody composition/bi-specific binding molecule of the present disclosure and therapeutic agent(s) to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

The anti-CD40 antibodies or antigen-binding portions thereof, antibody compositions, or bi-specific binding molecules of the present disclosure may be administered without additional therapeutic treatments, i.e., as a stand-alone therapy (monotherapy). Alternatively, treatment with the anti-CD40 antibodies or antigen-binding portions thereof, antibody compositions, or bi-specific binding molecules of the present disclosure may include at least one additional therapeutic treatment (combination therapy), e.g., another immunostimulatory agent, an anti-cancer agent (e.g., a chemotherapeutic agent, an anti-neoplastic agent, an anti-angiogenic agent, or a tyrosine kinase inhibitor), or a vaccine (e.g., a tumor vaccine).

In some embodiments, the antibody or antigen-binding portion thereof, antibody composition, or bi-specific binding molecule may be co-administered or formulated with another medication/drug for the treatment of cancer. The additional therapeutic treatment may comprise, e.g., a chemotherapeutic, anti-neoplastic, or anti-angiogenic agent, a different anti-cancer antibody, and/or radiation therapy.

Pharmaceutical articles comprising an anti-CD40 antibody or antigen-binding portion thereof, antibody composition, or bi-specific binding molecule described herein and at least one other agent (e.g., a chemotherapeutic, anti-neoplastic, or anti-angiogenic agent) may be used as a combination treatment for simultaneous, separate or successive administration in cancer therapy. The other agent may by any agent suitable for treatment of the particular cancer in question, for example, an agent selected from the group consisting of alkylating agents, e.g., platinum derivatives such as cisplatin, carboplatin and/or oxaliplatin; plant alkoids, e.g., paclitaxel, docetaxel and/or irinotecan; antitumor antibiotics, e.g., doxorubicin (adriamycin), daunorubicin, epirubicin, idarubicin mitoxantrone, dactinomycin, bleomycin, actinomycin, luteomycin, and/or mitomycin; topoisomerase inhibitors such as topotecan; antimetabolites, e.g., fluorouracil and/or other fluoropyrimidines; FOLFOX; osimertinib; cyclophosphamide; anthracycline; dacarbazine; gemcitabine; or any combination thereof. In some embodiments, the anti-CD40 antibody or antigen-binding portion thereof, antibody composition, or bi-specific binding molecule described herein reestablishes responsiveness to the other agent.

An anti-CD40 antibody or antigen-binding portion thereof, antibody composition, or bi-specific binding molecule of the present disclosure may also be used in combination with other anti-cancer therapies such as vaccines, cytokines, enzyme inhibitors, immunostimulatory compounds, and T cell therapies. In the case of a vaccine, it may be, e.g., a protein, peptide or DNA vaccine containing one or more antigens which are relevant for the cancer being treated, or a vaccine comprising dendritic cells along with an antigen. Suitable cytokines include, for example, IL-2, IFN-gamma and GM-CSF. An example of a type of enzyme inhibitor that has anti-cancer activity is an indoleamine-2,3-dioxygenase (IDO) inhibitor, for example, 1-methyl-D-tryptophan (1-D-MT). Also contemplated is adoptive T cell therapy, which refers to various immunotherapy techniques that involve expanding or engineering patients' own T cells to recognize and attack their tumors.

It is also contemplated that an anti-CD40 antibody or antigen-binding portion thereof, antibody composition, or bi-specific binding molecule of the present disclosure may be used in adjunctive therapy in connection with tyrosine kinase inhibitors. These are synthetic, mainly quinazoline-derived, low molecular weight molecules that interact with the intracellular tyrosine kinase domain of receptors and inhibit ligand-induced receptor phosphorylation, e.g., by competing for the intracellular Mg-ATP binding site.

The present disclosure also contemplates the use of sequences (e.g., the six CDR or VH and VL sequences) of an anti-CD40 antibody or antigen-binding portion described herein in the preparation of a chimeric antigen receptor, which may be for use in CAR-T technology.

It is understood that the antibodies and antigen-binding portions thereof, antibody compositions, and bi-specific binding molecules of the present disclosure may be used in a method of treatment as described herein, may be for use in a treatment as described herein, and/or may be for use in the manufacture of a medicament for a treatment as described herein.

### Dose and Route of Administration

The antibodies or antigen-binding portions thereof, antibody compositions, or bi-specific binding molecules of the present disclosure may be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the antibodies are being administered as a stand-alone treatment or in combination with one or more additional anti-cancer treatments.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the present disclosure are generally dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of the present disclosure may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining of appropriate dosages and regimens is well-known in the relevant art and would be understood to be encompassed by the skilled artisan once provided the teachings disclosed herein.

An effective amount for tumor therapy may be measured by its ability to stabilize disease progression and/or ameliorate symptoms in a patient, and preferably to reverse disease progression, e.g., by reducing tumor size. The ability of an antibody, antigen-binding portion, antibody composition, or bi-specific binding molecule of the present disclosure to inhibit cancer may be evaluated by in vitro assays, e.g., as described in the examples, as well as in suitable animal models that are predictive of the efficacy in human tumors. Suitable dosage regimens will be selected in order to provide an optimum therapeutic response in each particular situation, for example, administered as a single bolus or as a continuous infusion, and with possible adjustment of the dosage as indicated by the exigencies of each case.

The antibodies or antigen-binding portions thereof, antibody compositions, or bi-specific binding molecules of the present disclosure may be administered by any method for administering peptides, proteins or antibodies accepted in the art, and are typically suitable for parenteral administration. As used herein, “parenteral administration” includes any route of administration characterized by physical breaching of a tissue of a subject and administration through the breach in the tissue, thus generally resulting in the direct administration into the blood stream, into muscle, or into an internal organ. Parenteral administration thus includes, but is not limited to, administration by injection, by application through a surgical incision, by application through a tissue-penetrating non-surgical wound, and the like. In particular, parenteral administration is contemplated to include, but is not limited to, subcutaneous, intraperitoneal, intramuscular, intrasternal, intracisternal, intravenous, intraarterial, intrathecal, intraurethral, intracranial, intratumoral, and intrasynovial injection or infusions. Particular embodiments include the intravenous and the subcutaneous routes.

### Diagnostic Uses and Compositions

The antibodies and antigen-binding portions of the present disclosure also are useful in diagnostic processes (e.g., in vitro, ex vivo). For example, the antibodies and antigen-binding portions can be used to detect and/or measure the level of CD40 in a sample from a patient (e.g., a tissue sample, or a body fluid sample such as an inflammatory exudate, blood, serum, bowel fluid, saliva, or urine). Suitable detection and measurement methods include immunological methods such as flow cytometry, enzyme-linked immunosorbent assays (ELISA), chemiluminescence assays, radioimmunoassays, and immunohistology. The present disclosure further encompasses kits (e.g., diagnostic kits) comprising the antibodies and antigen-binding portions described herein.

### Articles of Manufacture and Kits

The present disclosure also provides articles of manufacture, e.g., kits, comprising one or more containers (e.g., single-use or multi-use containers) containing a pharmaceutical composition of an anti-CD40 antibody or antigen-binding portion thereof, composition, or bi-specific binding molecule described herein, optionally an additional biologically active molecule (e.g., another therapeutic agent), and instructions for use. The antibody or antigen-binding portion, composition, or bi-specific binding molecule, and optional additional biologically active molecule, can be packaged separately in suitable packing such as a vial or ampule made from non-reactive glass or plastic. In certain embodiments, the vial or ampule holds a concentrated stock (e.g., 2×, 5×, 10× or more) of the antibody or antigen-binding portion, composition, or bi-specific binding molecule and optionally the biologically active molecule. In certain embodiments, the articles of manufacture such as kits include a medical device for administering the antibody or antigen-binding portion, composition, or bi-specific binding molecule and/or biologically active molecule (e.g., a syringe and a needle); and/or an appropriate diluent (e.g., sterile water and normal saline). The present disclosure also includes methods for manufacturing said articles.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

In order that the present disclosure may be better understood, the following examples are set forth. These examples are for purposes of illustration only and are not to be construed as limiting the scope of the present disclosure in any manner.

### EXAMPLES

### Example 1. Cloning of Anti-CD40 Antibodies from Rat B Cells

Materials and Methods

Antibodies against human CD40 were isolated from an antibody repertoire derived from OmniRat® rats (Osborn et al., J Immunol. 190(4):1481-90 (2013)), a transgenic rat strain from Ligand Pharmaceuticals Inc. that produces antibodies with fully human idiotypes. Cloning of rat-derived antibody genes from single-cell sorted antibody-secreting B cells (ASC) was performed by means of Symplex™ antibody discovery technology (Meijer et al., J Mol Biol 358(3):764-72 (2006)).

Antibody repertoire constructs encoding fully human immunoglobulins in IgG₁-LALA format (see below) were transfected into HEK293 cells. Cell supernatants were screened for binding to CD40 expressed on the surface of CHO cells using flow cytometry in a high-throughput format. CD40 reactive clones were analyzed by DNA sequencing and antibody-encoding DNA sequences were extracted. Selected antibody clones were expressed and tested functionally as described below.

Missense mutations in the amino termini of heavy and light chains that were introduced by the use of degenerate primers in the Symplex™ cloning of the antibody-encoding cDNA fragments were corrected back to germline sequence. Table 1 shows the heavy and light chain variable domain nucleotide sequences of the germlined antibodies designated 17303, 16040, 15833, 16154, 15888, and 15948. The correction process involves amino terminal sequence correction to germline as well as codon usage optimization. The targets for matching to human germline sequences were identified by blast homology searches for the heavy chain and the light chain variable regions.

Protein sequences of the variable domains, the constant regions and the complementarity determining regions (CDRs) of antibodies 17303, 16040, 15833, 16154, 15888, and 15948 are shown in Table 2, Table 3, and Table 4, respectively.

### Results

Table 1 shows nucleotide sequences encoding the variable domains of antibodies 17303, 16040, 15833, 16154, 15888, and 15948.

TABLE 1
Variable domain nucleotide sequences of antibodies
17303, 16040, 15833, 16154, 15888, and 15948

| Ab | Sequence (5′ to 3′) |
|---|---|
| 17303 VH | GAGGTGCAGCTGGTGGAGAGCGGAGGAGGCCTGGTGCAGCCAGGCAGAAGCCT |
| SEQ ID NO: 1 | GAGGCTGTCTTGCGCCGCTTCCGGCTTCACCTTTGACGATCACGCCATGCATT |
| | GGGTGCGTCAAGCACCCGGCAAGGGCCTGGAGTGGGTGTCTGGCATCTCCTGG |
| | AACTCCGGCAGCATCGGCTACGCCGAGTCTGTGAAGGGCAGGTTCACCATCAG |
| | CCGGGACAACGCTAAGAATTCTCTGTATCTGCAAATGAATTCCCTGCGCGCCG |
| | AGGATACAGCTCTGTACTATTGTGCCAAGGACAGGCGGGGCCCATTTGATTAC |
| | TGGGGCCAGGGCACCCTGGTGACAGTCTCGAGT |
| 16040 VH | CAGGTGCAGCTGGTGGAGTCCGGAGGAGGAGTGGTGCAGCCAGGCAGGTCCCT |
| SEQ ID NO: 11 | GAGGCTGAGCTGCGCCGCTTCTGGCTTCACCTTTTCCATCTACGGAATGCACT |
| | GGGTGAGGCAGGCTCCTGGCAAGGGCCTGGAGTGGGTGGCTGTGATCTCCTAT |
| | GACGGCAGCAACAAGTACTATGCCGATAGCGTGAAGGGCAGATTCACCATCTC |
| | TCGCGACAACTCCAAGAATACACTGTACCTGCAGATGAACTCTCTGAGAGCCG |
| | AGGACACAGCCGTGTACTATTGTGCCGCTGGCAATTGGTTTGATCCATGGGGC |
| | CAGGGCACCCTGGTGACAGTCTCGAGT |
| 15833 VH | CAGGTGCAGCTGGTGGAGAGCGGAGGAGGAGTGGTGCAGCCAGGCAGGTCCCT |
| SEQ ID NO: 21 | GAGGCTGAGCTGCGCCGCTTCTGGCTTCACCTTTTCTAACTACGCCATGCACT |
| | GGGTGAGGCAGGCTCCTGGCAAGGGCCTGGAGTGGGTGGCCCTGATCTCCTTC |
| | GACGGCAGCTATAAGTACTATGCTGATTCTGTGAAGGGCAGGTTCACCATCTC |
| | CCGCGACAACTCAAAGAATACACTGTACCTGCAGATGAATTCCCTGAGAGCCG |
| | AGGACACAGCCGTGTACTATTGCGTGAAGGATGCCGGCTACTCCAGCTCTTGG |
| | TTCTTTGATTATTGGGGCCAGGGCACCCTGGTGACAGTCTCGAGT |
| 16154 VH | GAGGTGCAGCTGGTGGAGTCCGGAGGAGGCCTGGTGCAGCCAGGAGGCTCCCT |
| SEQ ID NO: 31 | GAGGCTGAGCTGCGCCGCTTCTGGCTTCACCTTTACATCCTACTGGATGAGCT |
| | GGGTGAGACAGGCTCCAGGCAAGGGCCTGGAGTGGGTGGCTAACATCAAGCAG |
| | GACGGCTCTGAGAAGTACTATGTGGATTCCGTGAAGGGCAGGTTCACCATCAG |
| | CCGGGACAACGCCAAGAATTCTCTGTATCTGCAAATGAATTCCCTGAGAGCCG |
| | AGGATACAGCCGTGTACTATTGTGCCCGCGGCCCAGTGGCTGGACCTTACTGG |
| | TATTTTGACCTGTGGGGCAGGGGCACCCTGGTGACAGTCTCGAGT |
| 15888 VH | CAGGTGCAGCTGCAGCAGTCCGGACCAGGCCTGGTGAAGCCTTCTCAGACCCT |
| SEQ ID NO: 41 | GTCCCTGACATGCGCCATCAGCGGCGACTCCGTGTCCTCCAACTCCGCCACCT |
| | GGAATTGGATCAGACAGTCTCCTTCCCGCGGCCTGGAGTGGCTGGGAAGGACA |
| | TACTATCGGTCTAAGTGGTACAACGACTATGCCGTGTCCGTGAAGAGCAGGAT |
| | CACCATCAACCCAGATACAAGCAAGAATCAGTTCTCTCTGCAGCTGAATTCCG |
| | TGACCCCCGAGGACACAGCCGTGTACTATTGTGCTAGAGGCGGACACTCCATC |
| | GCTGTGGCTGGACCATTTGATTACTGGGGCCAGGGCACCCTGGTGACAGTCTC |
| | GAGT |
| 15948 VH | CAGGTGCAGCTGGTGGAGTCTGGAGGAGGAGTGGTGCAGCCAGGCAGGTCCCT |
| SEQ ID NO: 51 | GAGGCTGTCCTGCGCCGCCTCCGGCTTCACCTTTTCCATCTACGGAATGCACT |
| | GGGTGAGGCAGGCTCCAGGCAAGGGCCTGGAGTGGGTGGCTGTGATCTCTTAT |
| | GACGGCTCCAACAAGTACTATGCCGATTCCGTGAAGGGCAGATTCACCATCAG |
| | CCGCGACAACTCTAAGAATACACTGTACCTGCAGATGAATAGCCTGAGAGCCG |
| | AGGATACAGCCGTGTACTATTGTGCTAAGGACCCCGATTACTATGGCTCCGGC |
| | AGCTACTATCCTGACTGGGGCCAGGGCACCCTGGTGACAGTCTCGAGT |
| 17303 VL | GACATCGTGATGACCCAGAGCCCTGATTCTCTGGCCGTGTCTCTGGGCGAGAG |
| SEQ ID NO: 2 | AGCTACAATCAACTGCAAGTCCAGCCAGTCCGTGCTGTCTTCCAGCAACAATA |
| | AGAATTACCTGGCCTGGTATCAGCAGAAGCCAGGCCAGCCCCCTAAGCTGCTG |
| | ATCTACTGGGCTTCCACCAGGGAGAGCGGAGTGCCAGACCGGTTCAGCGGCTC |
| | TGGCTCCGGCACAGACTTCACCCTGACAATCTCTTCCCTGCAAGCCGAGGACG |
| | TGGCCGTGTACTATTGTCAGCAGTACTATTCCACCCCACGCACATTCGGCCAG |
| | GGCACCAAGGTGGAGATCAAG |
| 16040 VL | GACATCCAGCTGACACAGAGCCCTTCTTTCCTGTCCGCCTCCGTGGGCGATAG |
| SEQ ID NO: 12 | GGTGACCATCACATGCCGGGCCTCTCAGGGCATCTCCAGCTACCTGGCTTGGT |
| | ATCAGCAGAAGCCCGGCAAGGCCCCTAAGCTGCTGATCTACGCTGCTTCTACC |
| | CTGCAGTCCGGAGTGCCAAGCAGATTCTCTGGCTCCGGCAGCGGCACAGAGTT |
| | TACCCTGACAATCTCTTCCCTGCAACCAGAGGACTTCGCCACCTACTATTGTC |
| | AGCAGCTGAACCGCTATCCCCTGACCTTTGGCGGCGGCACAAAGGTGGAGATC |
| | AAG |
| 15833 VL | GACATCCAGATGACCCAGTCCCCTTCCAGCGTGTCTGCTTCCGTGGGCGACAG |
| SEQ ID NO: 22 | GGTGACCATCACATGCCGGGCCAGCCAGGATATCTCTTCCTGGCTGGCTTGGT |
| | ACCAGCAGAAGCCCGGCAAGGCCCCTAAGCTGCTGATCTATGCTGCCTCCTCC |
| | CTGCAGAGCGGAGTGCCATCTAGGTTCAGCGGCTCTGGCTCCGGCACAGACTT |
| | TACCCTGACAATCTCCTCACTGCAGCCAGAGGATTTCGGCAACTACTATTGTC |
| | AGCAGGCCAATTCTTTCCCCTGGACCTTTGGCCAGGGCACAAAGGTGGAGATC |
| | AAG |
| 16154 VL | GCCATCCAGATGACCCAGAGCCCTTCCAGCCTGTCCGCCTCCGTGGGCGACAG |
| SEQ ID NO: 32 | GGTGACCATCACATGCCGGGCCTCTCAGGGCATCAGAAACGATCTGGGCTGGT |
| | ACCAGCAGAAGCCCGGCAAGGCTCCTAAGCTGCTGATCTATGCCGCTTCTTCC |
| | CTGCAATCTGGAGTGCCATCCCGCTTCTCTGGATCCGGAAGCGGAACCGACTT |
| | TACCCTGACAATCAGCTCTCTGCAGCCAGAGGACTTCGCCACATACTATTGTC |
| | TGCAGGATTACCACTATCCCCTGACCTTTGGCGGCGGCACAAAGGTGGAGATC |
| | AAG |
| 15888 VL | GACATCGTGATGACCCAGTCCCCTGATAGCCTGGCCGTGAGCCTGGGAGAGAG |
| SEQ ID NO: 42 | GGCTACAATCAACTGCAAGTCCAGCCAGTCTGTGCTGTACTCTTCCAACAATA |
| | AGAATTACCTGGCCTGGTATCAGCAGAAGCCAGGCCAGCCCCCTAAGCTGCTG |
| | ATCTATTGGGCTTCTACCAGGGACTCCGGAGTGCCAGATCGGTTCTCCGGAAG |
| | CGGATCTGGCACAGACTTTACCCTGACAATCAGCTCTCTGCAAGCCGAGGATG |
| | TGGCCGTGTACTATTGTCAGCAGTACTATAACTCCCCACCACCTACCTTCGGC |
| | CAGGGAACAAAGGTGGAGATCAAG |
| 15948 VL | GACATCCAGCTGACACAGAGCCCTTCTTTCCTGTCCGCCTCCGTGGGCGATAG |
| SEQ ID NO: 52 | GGTGACCATCACATGCCGGGCCTCTCAGGGCATCTCCAGCTACCTGGCTTGGT |
| | ATCAGCAGAAGCCCGGCAAGGCCCCTAAGCTGCTGATCTACGCTGCTTCTACC |
| | CTGCAGTCCGGAGTGCCAAGCAGGTTCTCTGGCTCCGGCAGCGGCACAGAGTT |
| | TACCCTGACAATCTCTTCCCTGCAACCAGAGGACTTCGCCACCTACTATTGTC |
| | AGCAGGTGAAGTCCTATCCCCTGACCTTTGGCGGCGGCACAAAGGTGGAGATC |
| | AAG |

Table 2 shows the deduced amino acid sequences of antibodies 17303, 16040, 15833, 16154, 15888, and 15948. CDRs are in boldface and underlined.

TABLE 2
Variable domain amino acid sequences of antibodies
17303, 16040, 15833, 16154, 15888, and 15948.

| Ab | Sequence (N-terminus to C-terminus) |
|---|---|
| 17303 VH | EVQLVESGGGLVQPGRSLRLSCAASGFTFDDHAMHWVRQAPGKGLEWVSGISW |
| SEQ ID NO: 3 | NSGSIGYAESVKGRFTISRDNAKNSLYLQMNSLRAEDTALYYCAKDRRGPFDY |
| | WGQGTLVTVSS |
| 16040 VH | QVQLVESGGGVVQPGRSLRLSCAASGFTFSIYGMHWVRQAPGKGLEWVAVISY |
| SEQ ID NO: 13 | DGSNKYYADSVKGRFTISRDNSKNTLYLQMNSLRAEDTAVYYCAAGNWFDPWG |
| | QGTLVTVSS |
| 15833 VH | QVQLVESGGGVVQPGRSLRLSCAASGFTFSNYAMHWVRQAPGKGLEWVALISF |
| SEQ ID NO: 23 | DGSYKYYADSVKGRFTISRDNSKNTLYLQMNSLRAEDTAVYYCVKDAGYSSSW |
| | FFDYWGQGTLVTVSS |
| 16154 VH | EVQLVESGGGLVQPGGSLRLSCAASGFTFTSYWMSWVRQAPGKGLEWVANIKQ |
| SEQ ID NO: 33 | DGSEKYYVDSVKGRFTISRDNAKNSLYLQMNSLRAEDTAVYYCARGPVAGPYW |
| | YFDLWGRGTLVTVSS |
| 15888 VH | QVQLQQSGPGLVKPSQTLSLTCAISGDSVSSNSATWNWIRQSPSRGLEWLGRT |
| SEQ ID NO: 43 | YYRSKWYNDYAVSVKSRITINPDTSKNQFSLQLNSVTPEDTAVYYCARGGHSI |
| | AVAGPFDYWGQGTLVTVSS |
| 15948 VH | QVQLVESGGGVVQPGRSLRLSCAASGFTFSIYGMHWVRQAPGKGLEWVAVISY |
| SEQ ID NO: 53 | DGSNKYYADSVKGRFTISRDNSKNTLYLQMNSLRAEDTAVYYCAKDPDYYGSG |
| | SYYPDWGQGTLVTVSS |
| 17303 VL | DIVMTQSPDSLAVSLGERATINCKSSQSVLSSSNNKNYLAWYQQKPGQPPKLL |
| SEQ ID NO: 4 | IYWASTRESGVPDRFSGSGSGTDFTLTISSLQAEDVAVYYCQQYYSTPRTFGQ |
| | GTKVEIK |
| 16040 VL | DIQLTQSPSFLSASVGDRVTITCRASQGISSYLAWYQQKPGKAPKLLIYAAST |
| SEQ ID NO: 14 | LQSGVPSRFSGSGSGTEFTLTISSLQPEDFATYYCQQLNRYPLTFGGGTKVEI |
| | K |
| 15833 VL | DIQMTQSPSSVSASVGDRVTITCRASQDISSWLAWYQQKPGKAPKLLIYAASS |
| SEQ ID NO: 24 | LQSGVPSRFSGSGSGTDFTLTISSLQPEDFGNYYCQQANSFPWTFGQGTKVEI |
| | K |
| 16154 VL | AIQMTQSPSSLSASVGDRVTITCRASQGIRNDLGWYQQKPGKAPKLLIYAASS |
| SEQ ID NO: 34 | LQSGVPSRFSGSGSGTDFTLTISSLQPEDFATYYCLQDYHYPLTFGGGTKVEI |
| | K |
| 15888 VL | DIVMTQSPDSLAVSLGERATINCKSSQSVLYSSNNKNYLAWYQQKPGQPPKLL |
| SEQ ID NO: 44 | IYWASTRDSGVPDRFSGSGSGTDFTLTISSLQAEDVAVYYCQQYYNSPPPTFG |
| | QGTKVEIK |
| 15948 VL | DIQLTQSPSFLSASVGDRVTITCRASQGISSYLAWYQQKPGKAPKLLIYAAST |
| SEQ ID NO: 54 | LQSGVPSRFSGSGSGTEFTLTISSLQPEDFATYYCQQVKSYPLTFGGGTKVEI |
| | K |

Table 3 shows heavy and light chain constant region amino acid sequences (CH and CL, respectively). “IgG₁ LALA” refers to the presence of “LALA” mutations in the heavy chain (L234A/L235A, numbered according to the IMGT® Eu numbering scheme) that are known to reduce effector function of the Fc region of IgG₁ antibodies (Hezareh et al., J Virol. 75(24):12161-68 (2001); Hessell et al., Nature 449(7158):101-04 (2007)).

TABLE 3
Constant region amino acid sequences of antibodies
17303, 16040, 15833, 16154, 15888, and 15948

| Fragment | Sequence (N-terminus to C-terminus) |
|---|---|
| IgG₁-LALA CH | ASTKGPSVFPLAPSSKSTSGGTAALGCLVKDYFPEPVTVSWNSGALTSGVHTF |
| added to the VH | PAVLQSSGLYSLSSVVTVPSSSLGTQTYICNVNHKPSNTKVDKRVEPKSCDKT |
| SEQ ID NO: 61 | HTCPPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSREEMTKNQVSLTCLVKGFYPSDIAVEWE |
| | SNGQPENNYKTTPPVLDSDGSFFLYSKLTVDKSRWQQGNVFSCSVMHEALHNH |
| | YTQKSLSLSPGK |
| Kappa CL added | RTVAAPSVFIFPPSDEQLKSGTASVVCLLNNFYPREAKVQWKVDNALQSGNSQ |
| to the VL | ESVTEQDSKDSTYSLSSTLTLSKADYEKHKVYACEVTHQGLSSPVTKSFNRGE |
| SEQ ID NO: 62 | C |

Table 4 shows heavy and light chain CDR amino acid sequences of antibodies 17303, 16040, 15833, 16154, 15888, and 15948, wherein the CDRs are defined according to the IMGT® system. SEQ ID NOs. of the sequences are shown in parentheses.

TABLE 4
CDR amino acid sequences of antibodies
17303, 16040, 15833, 16154, 15888, and 15948

| | Sequence (N-terminus to C-terminus) | | | | | |
|---|---|---|---|---|---|---|
| | H- | H- | H- | L- | L- | L- |
| Ab | CDR1 | CDR2 | CDR3 | CDR1 | CDR2 | CDR3 |
| 17303 | GFTFDDHA | ISWNSGSI | CAKDRRGPFDYW | QSVLSSSNNKNY | WAS | CQQYYSTPRTF |
| | (5) | (6) | (7) | (8) | (9) | (10) |
| 16040 | GFTFSIYG | ISYDGSNK | CAAGNWFDPW | QGISSY | AAS | CQQLNRYPLTF |
| | (15) | (16) | (17) | (18) | (19) | (20) |
| 15833 | GFTFSNYA | ISFDGSYK | CVKDAGYSSSWFFDYW | QDISSW | AAS | CQQANSFPWTF |
| | (25) | (26) | (27) | (28) | (29) | (30) |
| 16154 | GFTFTSYW | IKQDGSEK | CARGPVAGPYWYFDLW | QGIRND | AAS | CLQDYHYPLTF |
| | (35) | (36) | (37) | (38) | (39) | (40) |
| 15888 | GDSVSSNSAT | TYYRSKW | CARGGHSIAVAGPFDYW | QSVLYSSNNKNY | WAS | CQQYYNSPPPTF |
| | (45) | (46) | (47) | (48) | (49) | (50) |
| 15948 | GFTFSIYG | ISYDGSNK | CAKDPDYYGSGSYYPDW | QGISSY | AAS | CQQVKSYPLTF |
| | (55) | (56) | (57) | (58) | (59) | (60) |

Table 5 shows SEQ ID NO information for antibodies 17303, 16040, 15833, 16154, 15888, and 15948. Unless otherwise stated, the sequences are amino acid sequences.

TABLE 5
SEQ ID NOs for antibodies 17303, 16040, 15833, 16154, 15888, and 15948

| | VH | VL | VH | VL | H- | H- | H- | L- | L- | L- |
|---|---|---|---|---|---|---|---|---|---|---|
| Name | nt | nt | aa | aa | CDR1 | CDR2 | CDR3 | CDR1 | CDR2 | CDR3 |
| 17303 | 1 | 2 | 3 | 4 | 5 | 6 | 7 | 8 | 9 | 10 |
| 16040 | 11 | 12 | 13 | 14 | 15 | 16 | 17 | 18 | 19 | 20 |
| 15833 | 21 | 22 | 23 | 24 | 25 | 26 | 27 | 28 | 29 | 30 |
| 16154 | 31 | 32 | 33 | 34 | 35 | 36 | 37 | 38 | 39 | 40 |
| 15888 | 41 | 42 | 43 | 44 | 45 | 46 | 47 | 48 | 49 | 50 |
| 15948 | 51 | 52 | 53 | 54 | 55 | 56 | 57 | 58 | 59 | 60 |

nt: nucleotide
aa: amino acid

### Example 2. Measurement of Antibody Affinities Towards Human and Cynomolgus CD40

This example demonstrates the binding of anti-CD40 antibodies to human and cynomolgus CD40 extracellular domains as measured by surface plasmon resonance (SPR).

### Materials and Methods

The kinetic binding analysis was performed by Surface Plasmon Resonance (SPR), using a Continuous Flow Microspotter (CFM, Wasatch Microfluidics, Salt Lake City, US) combined with an IBIS MX96 SPR instrument (IBIS Technologies, The Netherlands).

His-tagged human CD40 extracellular domain (ECD) was purchased from Sino Biological, China. Binding kinetics were measured under monovalent antigen conditions by immobilizing anti-CD40 antibodies and keeping the monovalent CD40 antigen in solution. Antibodies were captured onto a G-a-hu-IgG Fc SensEye® (Ssens BV, The Netherlands) for 15 minutes using a Continuous Flow Microspotter (CFM, Wasatch Microfluidics, Salt Lake City, US). After spotting, the SensEye® was positioned in the IBIS MX96 biosensor and captured proteins were fixed to the surface using FixIT kit (Ssens BV, The Netherlands). Kinetic analysis was performed by applying kinetic titration series (Karlsson et al., Anal Biochem. 349(1):136-47 (2006)) having antigen injections as increasing concentrations from 0.41 nM to 300 nM. Antigen association was performed for 15 minutes and antigen dissociation was performed for 15 minutes. After each antigen injection series, the surface was regenerated with 100 mM H₃PO₄, pH 3 regeneration buffer. The recorded binding responses were fitted to a simple Langmuir 1:1 binding model with Scrubber 2 software for calculation of the on-rate (kₒₙ or kₐ), off-rate (k_{off} or k_{d}) and affinity (K_{D}) constants.

### Results

The results of the affinity measurement demonstrate that the evaluated antibodies 17303, 16040, 15833, 16154, 15888, and 15948 bind human and cynomolgus CD40 ECD with different affinities. The detailed binding kinetics are tabulated in Table 6 below.

TABLE 6
Binding kinetics of antibodies 17303,
16040, 15833, 16154, 15888, and 15948

| Antibody | CD40 ECD | kₒₙ (M−1 s−1) | k_{off} (s−1) | K_{D} (M) |
|---|---|---|---|---|
| 17303 | Human | 4.9E+04 | 3.8E−04 | 7.8E−09 |
| 17303 | Cynomolgus | 1.5E+05 | 1.4E−03 | 9.8E−09 |
| 16040 | Human | 5.2E+04 | 3.6E−04 | 7.0E−09 |
| 16040 | Cynomolgus | 8.1E+04 | 1.3E−03 | 1.6E−08 |
| 15833 | Human | 4.1E+04 | 1.2E−04 | 2.8E−09 |
| 15833 | Cynomolgus | 8.0E+04 | 1.2E−04 | 1.5E−09 |
| 16154 | Human | 5.3E+04 | 3.1E−04 | 5.7E−09 |
| 16154 | Cynomolgus | 1.1E+05 | 5.1E−04 | 4.6E−09 |
| 15888 | Human | 1.9E+05 | 1.4E−04 | 7.5E−10 |
| 15888 | Cynomolgus | 3.3E+05 | 1.4E−04 | 4.4E−10 |
| 15948 | Human | 9.9E+04 | 3.1E−04 | 3.1E−09 |
| 15948 | Cynomolgus | 3.7E+04 | 2.1E−03 | 5.7E−08 |

### Example 3. Cloning of Anti-CD40 Reference Antibody Analogues

### Materials and Methods

The amino acid sequences encoding the heavy and light chain variable domains of the antibody analogues in Table 7 were obtained from the listed patents or patent applications. The protein sequences were reverse translated to DNA sequences with human codon usage. The corresponding DNA sequences were gene synthesized and cloned into expression vectors containing human heavy or light chain constant regions, resulting in expression of full-length antibody chains. The human antibody isotype selected for expression is listed in the antibody format column. CHO cells were transfected with the resulting expression plasmids using a standard protein expression system. The corresponding antibody supernatants were purified using standard protein A purification column chromatography.

TABLE 7
Listing of gene-synthesized antibody analogues
and the corresponding antibody format

| | Antibody | |
|---|---|---|
| Antibody | format | Source |
| selicrelumab | IgG₂/kappa | U.S. Pat. Pub. 2018/US9856319 |
| analogue | | B2 (SEQ ID NOs: 71 and 49) |
| ADC-1013 | IgG₁/lambda | U.S. Pat. Pub. 2017/9676862 |
| analogue | | B2 (SEQ ID NOs: 62 and 25) |

### Example 4. Direct Binding of Anti-CD40 Antibodies to CHO-S Cells Transfected with Human or Cynomolgus CD40 Protein

This example demonstrates the binding of anti-CD40 antibodies to human and cynomolgus CD40 protein transiently expressed on cells.

### Materials and Methods

The binding of six anti-CD40 antibodies to human or cynomolgus CD40 protein expressed on CHO-S cells was evaluated and compared to that of selicrelumab or an ADC-1013 analogue. The anti-CD40 antibodies were incubated with hamster CHO-S cells transiently expressing human or cynomolgus CD40 for 30 minutes at 4° C. The cells were washed twice and subsequently incubated for an additional 20 minutes with AF647-conjugated secondary anti-human IgG (H+L) antibody. After the washing step, antibody binding was detected using the high-throughput flow cytometer iQue Screener PLUS (Sartorius) measuring the GeoMean of AF647 signal in each well. Every concentration was assayed in triplicate and a 12-point titration curve was generated for each antibody.

### Results

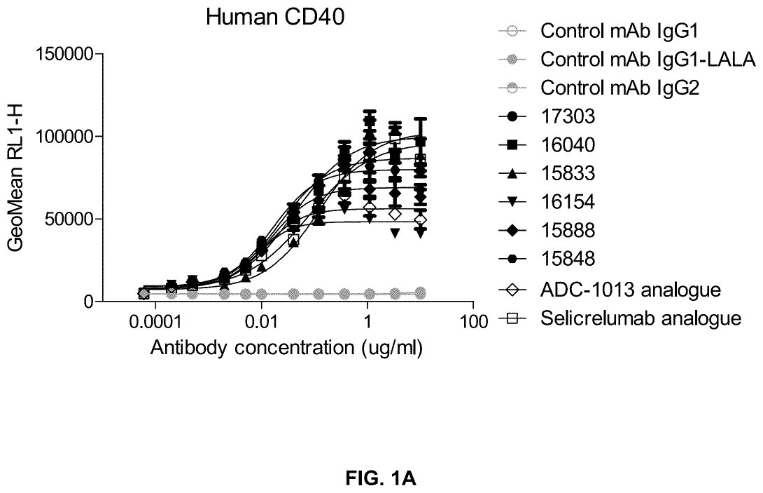

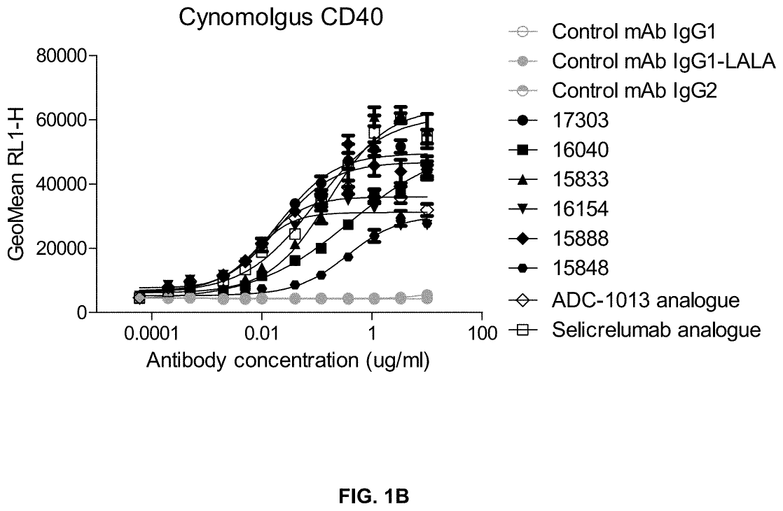

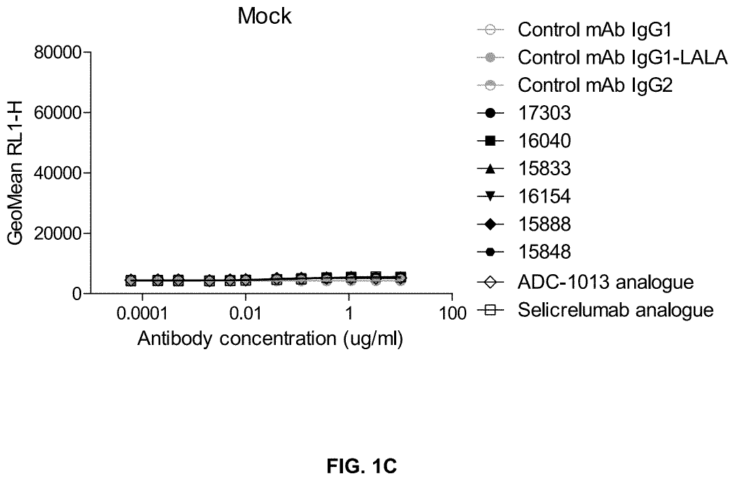

The binding curves of the antibodies to human or cynomolgus CD40 expressed on cells are shown in FIGS. 1A-1C. The assayed antibodies all bound to cell-displayed human and cynomolgus CD40 protein, but with different potency and efficacy.

### Example 5. Identification of Anti-CD40 Antibody Mixtures with Superior Activity to Single Anti-CD40 Antibodies in a CD40-NFkB-Luciferase Reporter Assay

This example describes in vitro functional screening of a panel of anti-CD40 monoclonal antibodies and antibody mixtures in a CD40-NFkB-luciferase reporter assay with the purpose of identifying mixtures with superior activity to single antibodies.

### Materials and Methods

The activity of anti-CD40 mAb mixtures was investigated using a CD40-NFkB-luciferase reporter assay. The activity of a panel of 37 monoclonal antibodies in 2-antibody mixtures in all combinations (666 mixtures in total) was measured and compared to the activity of the corresponding single antibodies. 5 μg/mL of each anti-CD40 mAb were mixed resulting in a total mixture concentration of 10 μg/mL. The single antibodies were also tested at a concentration of 10 μg/mL.

The CD40-NFkB-luciferase cells were purchased from Promega (CD40 Bioassay kit). The reporter cells were plated approximately 24 hours prior to stimulation start and the activity was evaluated after 4 hours of stimulation with the indicated concentrations of antibodies or antibody mixtures. Activity was measured by adding Bio-Glo luciferase reagent to the cells and bioluminescence was measured using an Envision plate reader.

### Results

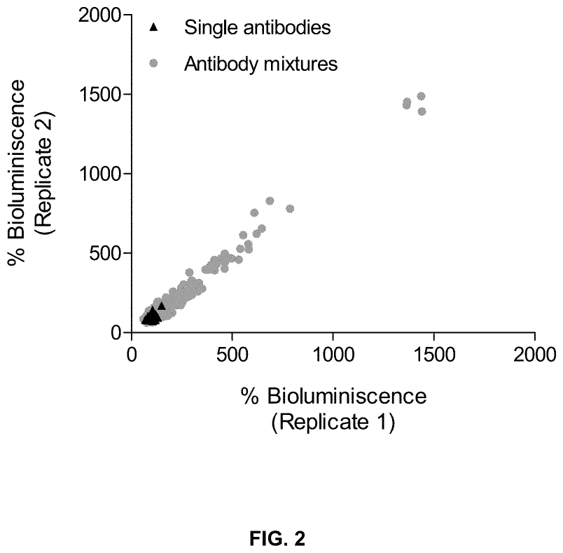

The ability of single anti-CD40 antibodies and antibody mixtures to stimulate CD40 activity in a CD40-NFkB-luciferase reporter assay is shown in FIG. 2. It is evident that the stimulatory function of the anti-CD40 antibodies is enhanced in the mixtures compared to the single antibodies. Thus, this example demonstrates that the mixtures are superior to the single antibodies.

### Example 6. Identification of Anti-CD40 Antibody Mixtures with Superior Activity to Single Anti-CD40 Antibodies in a B Cell Proliferation Assay

This example describes in vitro functional screening of a panel of anti-CD40 monoclonal antibodies and antibody mixtures in a B cell proliferation assay, with the purpose of identifying mixtures with superior activity to single antibodies.

### Materials and Methods

The activity of anti-CD40 mAb mixtures was investigated using a B cell proliferation assay. CD19⁺ B cells were isolated from peripheral blood mononuclear cells (PBMCs) from healthy donor material and cultured in the presence of IL-21 (50 ng/mL) and anti-CD40 antibodies. The activity of a panel of 37 mAbs in 2-antibody mixtures in all combinations (666 mixtures in total) was measured and compared to the activity the corresponding single antibodies. 5 μg/mL of each anti-CD40 mAb were mixed resulting in a total mixture concentration of 10 μg/mL. The single mAbs were also tested at a concentration of 10 μg/mL. The level of proliferation was measured by CellTiter-Glo® after 5 days of culture. Bioluminescence was measured using an Envision plate reader.

### Results

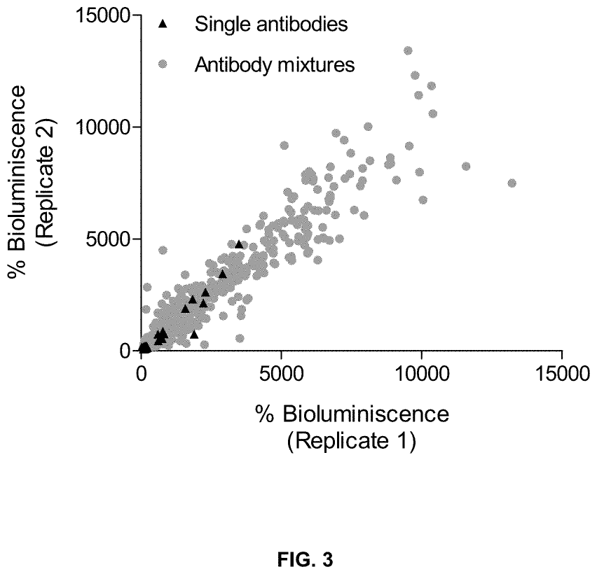

The ability of single anti-CD40 antibodies and antibody mixtures to stimulate proliferation of human primary B cells is shown in FIG. 3. It is evident that the stimulatory function of the anti-CD40 antibodies is enhanced in the mixtures compared to the single antibodies. Thus, this example demonstrates that the mixtures are superior to the single antibodies

### Example 7. Functionality of Anti-CD40 Antibodies in a CD40-NFkB-Luciferase Assay

This example describes in vitro functional evaluation of selected anti-CD40 monoclonal antibodies and antibody mixtures. The antibodies were evaluated for their ability to stimulate CD40 activity in a NFkB-reporter cell line. Analogues of selicrelumab and ADC-1013 were included for comparison.

### Materials and Methods

The ability of anti-CD40 antibodies (six single mAbs and five mixtures) to stimulate CD40 activity was measured in a CD40-NFkB-luciferase reporter assay and compared to analogues of selicrelumab and ADC-1013. The CD40-NFkB-luciferase cells were purchased from Promega (CD40 Bioassay kit). The reporter cells were plated approximately 24 hours prior to stimulation start and the activity was evaluated after 4 hours of stimulation with the indicated concentrations of antibodies or antibody mixtures. Activity was measured by adding Bio-Glo luciferase reagent to the cells and bioluminescence was measured using an Envision plate reader.

### Results

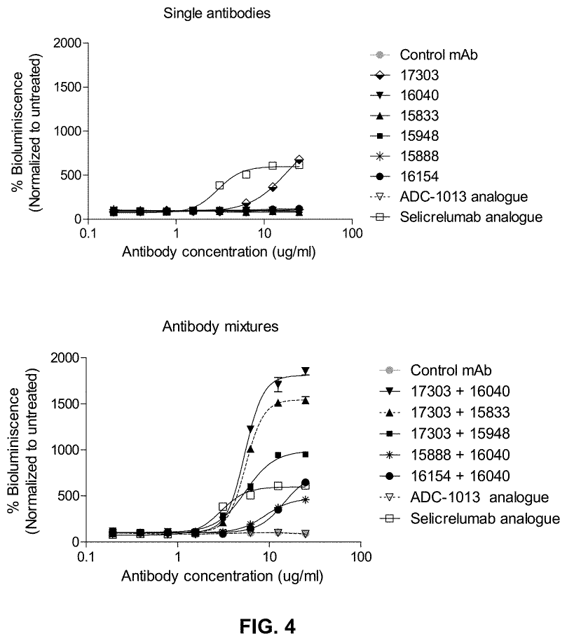

The stimulation of CD40 activity after treatment with the different anti-CD40 antibodies is shown in FIG. 4. It is evident that the stimulatory function of anti-CD40 antibodies in the CD40-NFkB-luciferase reporter assay is concentration dependent and that all of the antibody mixtures stimulate activity, albeit with different potency and efficacy. Moreover, this example demonstrates that the antibody mixtures are superior to the single mAbs in this assay.

### Example 8. Functionality of Anti-CD40 Antibodies in a CD40-NFkB-Luciferase Assay in the Presence of CD40 Ligand

This example describes in vitro functional evaluation of selected anti-CD40 monoclonal antibodies and antibody mixtures with the purpose of testing whether they inhibit CD40 ligand activity. The antibodies were evaluated for their CD40 ligand blocking activity in a CD40-NFkB-reporter cell line. Analogues of selicrelumab and ADC-1013 were included for comparison.

### Materials and Methods

The ability of anti-CD40 antibodies (six single mAbs and five mixtures) to inhibit CD40 ligand activity was measured in a CD40-NFkB-luciferase reporter assay in the presence of CD40 ligand (0.2 μg/mL, R&D systems (6420-CL/CF)) and compared to analogues of selicrelumab and ADC-1013. All antibodies and antibody mixtures were tested at a concentration of 10 μg/mL.

The CD40-NFkB-luciferase cells were purchased from Promega (CD40 Bioassay kit). The reporter cells were plated approximately 24 hours prior to stimulation start and the activity was evaluated after 4 hours of stimulation. Activity was measured by adding Bio-Glo luciferase reagent to the cells and bioluminescence was measured using an Envision plate reader.

### Results

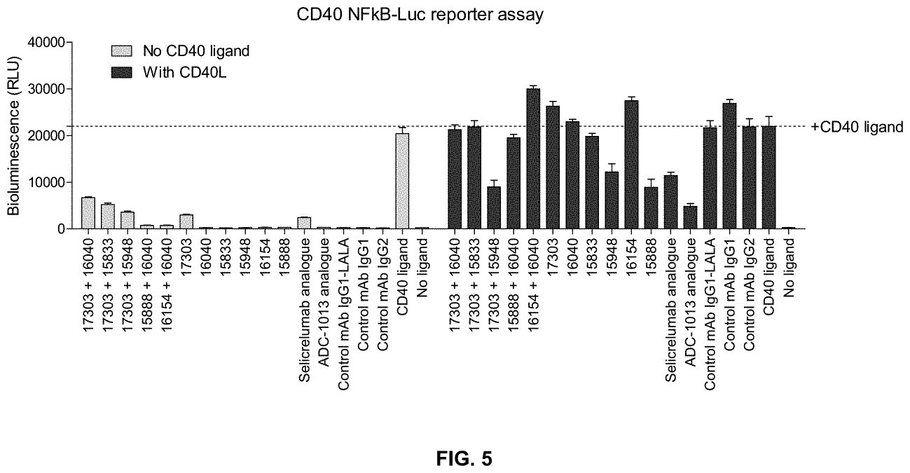

The stimulation of CD40 activity after treatment with the different anti-CD40 antibodies with and without CD40 ligand is shown in FIG. 5. Antibody mixtures 17303+16040, 17303+15833, 15888+16040 and 16154+16040, as well as monoclonal antibodies 17303, 16040, 15833 and 16154, do not inhibit the activity of the CD40 ligand. Antibody mixture 17303+15948 and monoclonal antibodies 15948 and 15888, as well as the analogues of selicrelumab and ADC-1013, show a stronger inhibition of CD40 ligand activity.

### Example 9. Functionality of Anti-CD40 Antibody Mixtures in a One-Way MLR Assay

### Materials and Methods

The ability of five anti-CD40 antibody mixtures to stimulate enhanced activity in a one-way mixed lymphocyte reaction (MLR) assay was evaluated and compared to analogues of selicrelumab and ADC-1013. In the one-way MLR assay, dendritic cells (DCs) and CD4⁺ T cells isolated from two different healthy donors are co-cultured to induce an alloantigen specific reaction resulting in cytokine production and T cell activation/proliferation. Dendritic cells (DCs) were differentiated from CD14⁺ monocytes by 9 days of culture with 20 ng/mL granulocyte-macrophage colony-stimulating factor (GM-CSF) and 20 ng/mL interleukin-4 (IL-4), and mixed in a 1:10 ratio with CD4⁺ T cells isolated from peripheral blood mononuclear cells (PBMCs) from healthy donor material. After 5 days of culture, supernatants were harvested and IFN-γ levels were determined by ELISA.

### Results

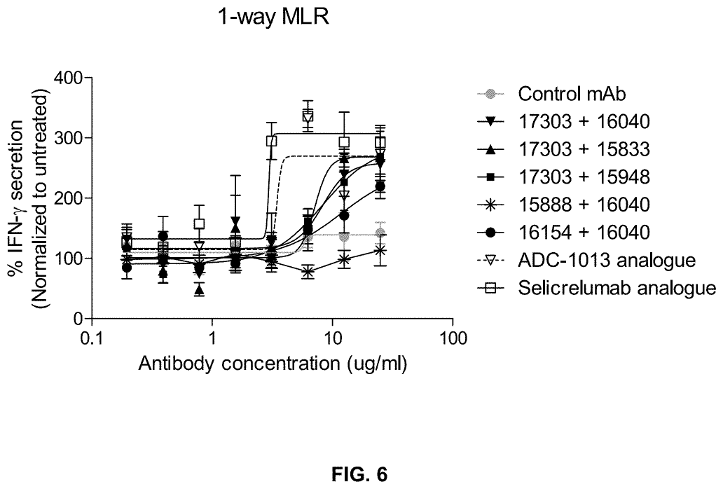

The effect of the different anti-CD40 antibodies in the one-way MLR is shown in FIG. 6. It is evident that the stimulatory function of anti-CD40 antibodies in the one-way-MLR assay is concentration dependent and that all the mixtures, except for 15888+16040, show agonistic activity, albeit with different potency and efficacy.

### Example 10. Functionality of Anti-CD40 Antibodies in a B Cell Proliferation Assay

This example describes the in vitro functional evaluation of selected anti-CD40 monoclonal antibodies and antibody mixtures. The antibodies were evaluated for their ability to stimulate proliferation of human B cells. Analogues of selicrelumab and ADC-1013 were included for comparison.

### Materials and Methods

The ability of anti-CD40 antibodies (six single mAbs and five mixtures) to stimulate B cell proliferation was evaluated and compared to analogues of selicrelumab and ADC-1013. CD19⁺ B cells were isolated from peripheral blood mononuclear cells (PBMCs) from healthy donor material and cultured in the presence of IL-21 (50 ng/mL) and anti-CD40 antibodies. The level of proliferation was measured using CellTiter-Glo® after 5 days of culture. Bioluminescence was measured using an Envision plate reader.

### Results

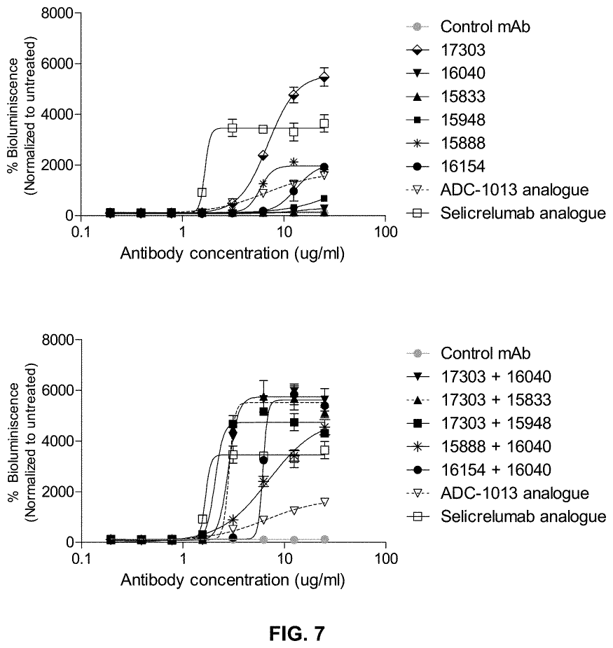

The stimulation of B cell proliferation after treatment with the different anti-CD40 antibodies is shown in FIG. 7. It is evident that the stimulatory function of anti-CD40 antibodies in the B cell proliferation assay is concentration dependent and that all antibody mixtures stimulate activity, albeit with different potency and efficacy. Moreover, this example demonstrates that the mixtures are superior to the single mAbs in this assay.

### Example 11. Functionality of Anti-CD40 Antibodies in a Dendritic Activation Assay

This example describes in vitro functional evaluation of selected anti-CD40 monoclonal antibodies and antibody mixtures. The antibodies were evaluated for their ability to stimulate cytokine secretion from human monocyte derived dendritic cells. Analogues of selicrelumab and ADC-1013 were included for comparison.

### Materials and Methods

The ability of anti-CD40 antibodies (six single mAbs and five mixtures) to stimulate dendritic cell activation was evaluated and compared to analogues of selicrelumab and ADC-1013. Dendritic cells (DCs) were differentiated from CD14⁺ monocytes by 8 days of culture with 20 ng/mL GM-CSF and 20 ng/mL IL-4. The DCs were activated by stimulating CD40 with anti-CD40 antibodies. After 48 hours of culture, DC activation was determined by harvesting supernatants and measuring IL-12p40 levels with ELISA.

### Results

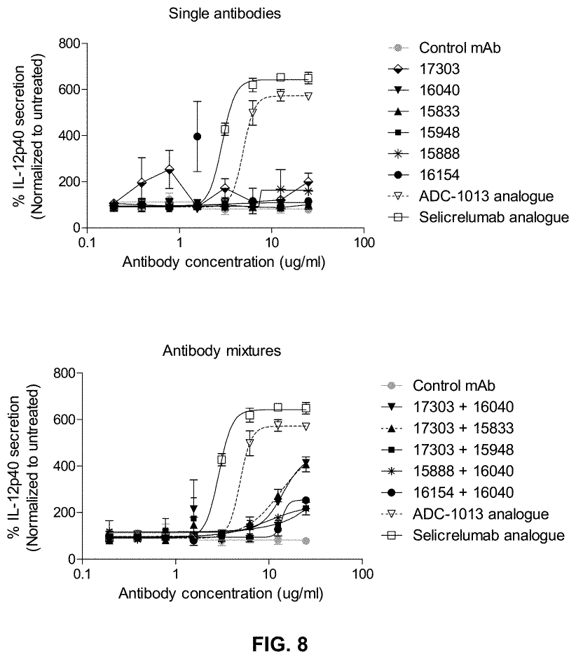

The stimulation of dendritic cell activation after treatment with the different anti-CD40 antibodies is shown in FIG. 8. It is evident that the stimulatory function of anti-CD40 antibodies in the dendritic activation assay is concentration dependent and that all of the antibody mixtures stimulate activity, albeit with different potency and efficacy. Moreover, this example demonstrates that the mixtures are superior to the single antibodies in this assay.

### Example 12. Effect of Anti-CD40 Antibodies on Proliferation of B Cells from Isolated Splenocytes from CD40 HuGEMM Mice

This example demonstrates the effect of selected anti-CD40 antibodies on proliferation of B cells from splenocytes isolated from a human CD40 knock-in mouse model.

### Materials and Methods

The ability of an anti-CD40 antibody mixture (17303+16040) to stimulate B cell proliferation was evaluated and compared to a selicrelumab analogue and CD40L. Mouse CD19⁺ B cells were isolated from the spleen of CD40 knock in-mice (CD40 HuGEMM; CrownBio, China) and cultured in the presence of IL-21 (50 ng/mL) and either CD40L or the indicated anti-CD40 antibod(ies). The level of proliferation was measured using CellTiter-Glo after five days of culture. Bioluminescence was measured using an Envision plate reader.

### Results

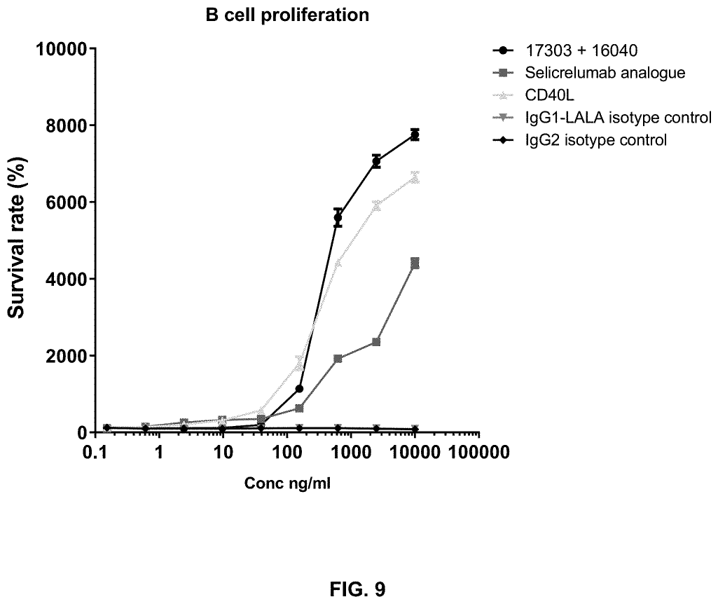

Stimulation of B cell proliferation after treatment with an anti-CD40 antibody mixture (17303+16040), selicrelumab analogue, or CD40L is shown in FIG. 9. The results demonstrate that the stimulatory function of the anti-CD40 antibodies in the B cell proliferation assay is concentration dependent, and that the anti-CD40 antibody mixture (17303+16040) is superior to the selicrelumab analogue and to CD40L.

### Example 13. In Vivo Efficacy of an Anti-CD40 Antibody Mixture in a Human Xenograft Tumor Model in CD34-NOG Mice

This example demonstrates the in vivo efficacy of an anti-CD40 antibody mixture in CD34-humanized mice engrafted with human Burkitt's lymphoma cells.

### Materials and Methods

CD34-humanized NOG (huCD34-NOG) mice were subcutaneously engrafted with Raji cells (a human Burkitt's lymphoma cell line). Treatment was initiated at the day of tumor cell injection and the mice were treated twice weekly for a total of six treatments by intraperitoneal injection of vehicle buffer or an anti-CD40 antibody mixture (17303+16040) at 5 mg/kg (n=4-5/group). Tumors were measured three times weekly by caliper in two dimensions, and tumor volume in mm³ was calculated per the formula: (width)²×length×0.5. Two-way ANOVA with Bonferroni's multiple comparisons test was applied to compare tumor volumes at each timepoint between treatment groups. Statistical analyses were performed using GraphPad Prism version 5.0 (GraphPad Software, Inc.).

### Results

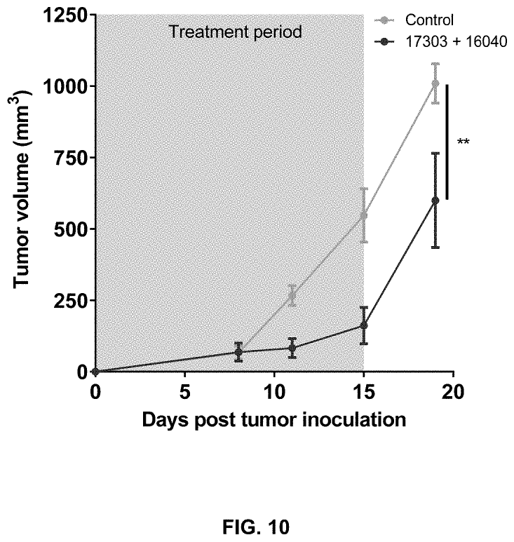

As shown in FIG. 10, treatment with the anti-CD40 antibody mixture resulted in significant tumor growth delay (P<0.01 vs. vehicle control) in mice reconstituted with human immune cells.

### Example 14. In Vivo Efficacy and Toxicity Evaluation of an Anti-CD40 Antibody Mixture in a Syngeneic MC38-OVA Tumor Model in CD40 HuGEMM Mice

This example demonstrates in vivo efficacy with no toxicity of an anti-CD40 antibody mixture in CD40 huGEMM mice (CrownBio, China) engrafted with murine MC38-OVA colon cancer cells.

### Materials and Methods

CD40 HuGEMM mice (a human CD40 knock-in mouse model; CrownBio, China) were subcutaneously engrafted with murine MC38-OVA colon carcinoma cells. Treatment was initiated when the tumors reached a volume of 50 mm³. The mice were treated twice weekly for a total of six treatments by intraperitoneal injection of vehicle buffer, anti-CD40 antibody mixture (17303+16040) at 20 mg/kg, or a selicrelumab analogue at 0.3 mg/kg (n=10/group). Tumors were measured twice weekly by caliper in two dimensions, and tumor volume in mm³ was calculated per the formula: (width)²×length×0.5. Mice were weighed twice weekly, and body weight change during treatment was compared to body weight at treatment initiation.

Two-way ANOVA with Bonferroni's multiple comparisons test was applied to compare tumor volumes at each timepoint between treatment groups. Statistical analyses were performed using Graph Pad Prism version 5.0 (GraphPad Software, Inc.).

### Results

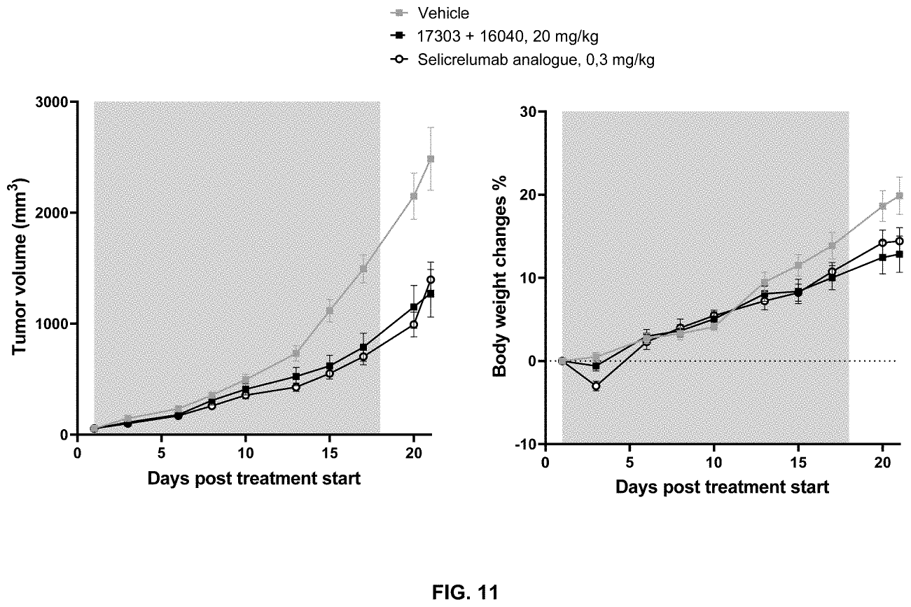

As shown in FIG. 11, the anti-CD40 antibody mixture and the selicrelumab analogue demonstrated a similar dose-dependent anti-tumor effect in MC38-OVA syngeneic tumors engrafted in CD40 HuGEMM mice. Treatment induced a significant tumor growth delay (P<0.05 vs. vehicle control). Initial body weight loss was observed when the mice were treated with the selicrelumab analogue. The mice recovered during the study. By contrast, no toxicity was observed based on body weight measurements for mice treated with the anti-CD40 antibody mixture.

### Example 15. Epitope Binning of Anti-CD40 Antibodies

This example describes the grouping of anti-CD40 antibodies into epitope bins based on paired competition patterns. Antibodies belonging to different epitope bins recognize different epitopes on the CD40 ECD.

### Materials and Methods

Investigation of paired antibody competition was performed by SPR using an IBIS-MX96 instrument (IBIS, Netherlands). Anti-CD40 antibodies were diluted to 3.25 μg/mL in PBS and spotted onto a G-a-hu-IgG Fc SensEye® by capturing for 15 minutes using a Continuous Flow Microspotter, followed by blocking of residual binding sites by Herceptin (trastuzumab) and chemical cross-linking by SensEye Fixlt kit (IBIS, Netherlands). After sensor preparation, antibody competition analysis was performed using a classical sandwich assay. Recombinant CD40-his ECD antigen was diluted in PBS, 0.05% Tween 20, and 200 nM Herceptin running buffer, injected at a 100 nM concentration and captured by the conjugated array of anti-CD40 antibodies. Next, individual injections of each of the anti-CD40 antibodies diluted to 100 nM in running buffer were performed to establish antibody competition patterns. Recombinant CD40 ligand (100 nM) was included as an analyte to characterize ligand blocking antibodies. Data was analyzed by Epitope Binning 2.0 (Wasatch, USA).

### Results

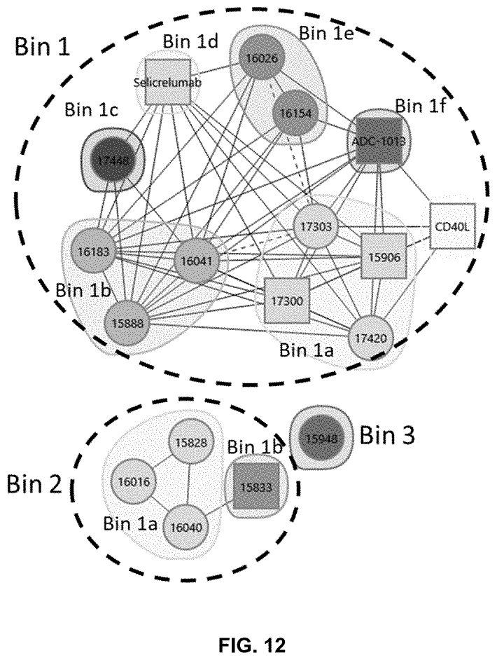

The competition pattern of 17 anti-CD40 antibodies is presented in FIG. 12. The tested antibodies grouped into three main epitope bins: 1, 2, and 3.

Bin 1 could be divided into several sub-bins: 1a (15906, 17300, 17303, and 17420), 1b (15888, 16041, and 16183), 1c (17448), 1d (selicrelumab analogue), 1e (16026 and 16154), and 1f (ADC-1013 analogue), indicating that the antibodies in bin 1 engaged overlapping epitopes. Bin 1 included both the selicrelumab and ADC-1013 analogues (in sub-bins 1d, and 1f, respectively), which did not cross-block. Further, the ADC-1013 analogue competed with CD40 ligand (CD40L) for binding, whereas the selicrelumab analogue did not. Selicrelumab and ADC-1013 are both known to bind epitopes located within cysteine rich domain 1 (CRD1) at the N terminus of CD40 (Yu et al., Cancer Cell 33: 664-675 (2018), and PCT Patent Publication WO 2013/034904). Selicrelumab engages an epitope on the opposite face of CD40 to the CD40-ligand binding site (Yu et al., supra). The anti-CD40 antibodies placed in bin 1 all cross-compete with either the selicrelumab analogue or the ADC-1013 analogue, indicating that antibodies placed in bin 1 have overlapping or closely adjacent epitopes located at CRD1 of CD40.

Bin 2 included four antibodies, and could be divided into two sub-bins: 2a (15828, 16016, and 16040) and 2b (15833). Antibodies in bin 2 only cross-blocked each other, indicating an epitope further down the receptor.

Bin 3 consisted of only one antibody, 15948, which did not compete with any of the other tested antibodies, suggesting an epitope separated from that of the rest of the tested antibodies.

In conclusion, antibodies in sub-bins 1a-1c and 1e engage unique epitopes that overlap with the epitopes of the selicrelumab and ADC-1013 analogues in sub-bins 1d and 1f, respectively, and the epitopes are located at CRD1 in the N-terminal end of CD40. The epitopes of antibodies in bins 2 and 3 do not overlap with the epitopes of the selicrelumab and ADC-1013 analogues.

### Example 16. Epitope Mapping of Anti-CD40 Antibodies by Mutagenesis and Surface Plasmon Resonance

Linear and conformational epitopes were characterized using a mutagenesis approach and surface plasmon resonance (SPR). This example illustrates how the epitopes recognized by monoclonal anti-CD40 antibodies 17303, 16040, and 15833 are distributed on the CD40 extracellular domain (ECD) and how they differentiate from the epitope of the selicrelumab analogue.

### Materials and Methods

The protein sequences of human, and rat (Rattus norvegicus) CD40 were downloaded from UniProt (Accession Nos. P25942, Q4QQW2 respectively) and aligned. To map linear epitopes, human CD40 ECD Fc fusion proteins were generated having 10 amino acids sequentially exchanged with corresponding rat CD40 sequence in segments overlapping by five amino acids. Conformational epitopes were characterized by alanine scanning mutagenesis of CD40 ECD.

The cDNA coding for human CD40 ECD was synthesized and cloned into a vector containing CMV promoter and human Ig Fc sequence (residues P101-K330), resulting in fusion of Ig Fc to the CD40 ECD C-terminus. Wild type (wt) and mutated human CD40 Fc fusion constructs were generated by standard gene synthesis techniques and proteins were expressed transiently in an ExpiCHO™ expression system. After harvesting, supernatants were tested for binding to anti-CD40 Fabs by surface plasmon resonance (SPR) using the Carterra® LSA™ Platform (Carterra, USA). An HC200M (Carterra, USA) chip was functionalized with goat anti-human Ig Fc (Southern Biotech) by amine-coupling. The chip was activated with freshly prepared 0.4 M EDC, 0.1 M sulfo-NHS, and 0.1 M MES, pH 5.5 (1:1:1 v/v/v) for 5 minutes, coupled with 75 μg/mL anti-human Ig Fc in 10 mM sodium acetate, pH 4.5, for 10 minutes, and excess reactive esters were quenched for 3 minutes by injection of 1 M ethanolamine, pH 8.5. The instrument was primed in running buffer (PBS pH 7.4, 0.01% Tween 20, 0.5 mg/mL BSA).

After priming and washing, CD40 fusion proteins in culture supernatants were captured onto individual spots of the chip for 12 minutes as duplicates. Fab analytes were each prepared in running buffer. Kinetic analysis was performed by applying a kinetic titration series of monomeric Fabs as increasing concentrations from 0.8 nM and up to 300 nM. Fab association was performed for 15 minutes and antigen dissociation was recorded for 15 minutes. After each cycle of Fab injections, the surface was regenerated by 0.45% H₃PO₄ for 2×20 s and washed for 5 min in running buffer.

Binding responses were analyzed in Carterra's KIT software tool by referencing to spots of wt rat CD40, buffer blanked and aligned to y-axis. Processed data was fitted to a simple Langmuir 1:1 binding model for calculation of the on-rate (k₀ or kₐ), off-rate (k_{off} or k_{d}) and affinity (K_{D}) constants. Mutations generating inactive proteins common for all antibodies were deselected and a 5-fold affinity reduction compared to the K_{D} of antibodies binding to wild type CD40 was used to define the epitopes.

### Results

The linear and conformational epitopes of anti-CD40 antibodies 17303, 16040, and 15833 and the selicrelumab analogue were mapped using chimeric receptor constructs with 10-amino acid segments replaced by rat CD40 sequence, or by alanine scanning, respectively. A cutoff of at least a 5-fold decrease in binding affinity compared to binding to wild type human CD40 was used to identify amino acids causing a significant loss of binding, thereby defining the epitopes (Table 8). Linear epitopes were defined as 10-amino acid segments of the human CD40 sequence that were replaced by rat CD40 sequence, with 5-amino acid overlap between segments. Contact residues were defined as single amino acids mutated to alanine.

TABLE 8
Summary of Epitopes for 17303, 16040,
15833, and a selicrelumab analogue

| Antibody | Linear epitopes | Contact Residues |
|---|---|---|
| 17303 | 31-65 | N34, S49, E53-T55, E58 |
| 16040 | 106-115, 131-145 | H110, H122, C125, G128-V131, |
| | | Q133-V138, D140, I142 |
| 15833 | 106-145 | H110, E114, R123, C125, G128- |
| | | Q133, A135, T136, V138, D140, I142 |
| Selicrelumab | 26-45 | R27-Q30, I33 |

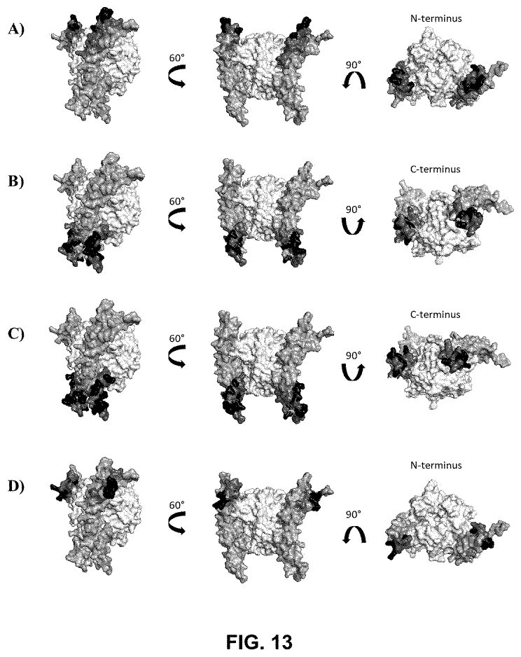

The extracellular domain of CD40 has an elongated structure of four cysteine-rich domains (CRDs), CRD1-4, each stabilized by 1-2 disulfide bridges (Hyun-Jung et al., J Biol Chem 286:11226-11235 (2011)). The identified linear epitopes and contact residues of antibodies 17303, 16040, 15833, and the selicrelumab analogue were mapped on the crystal structure of CD40 CRD 1-3 in complex with the CD40 ligand, CD154 (PDB: 3QD6, FIG. 13). The epitopes recognized by 17303 and the selicrelumab analogue were located at CRD1 at the top of the CD40 receptor. Antibody 17303 and the selicrelumab analogue were found to bind discrete but closely adjacent epitopes on each side of CRD1. The defined epitopes are in line with the epitope binning data in Example 15, placing the two antibodies in distinct sub-bins with closely related competitive binding profiles. Further, the epitope of the selicrelumab analogue is in agreement with the literature (Yu et al., supra).

The epitopes of antibodies 16040 and 15833 were located at CRD3 of CD40 (FIG. 13). The two antibodies bind very similar epitopes indicating that antibodies binding to this CDR3 epitope are favorable in a monoclonal antibody mixture in combination with a CRD1 binding antibody to induce CD40 clustering and agonistic activity.

In conclusion, the epitope mapping showed that the monoclonal antibodies of the investigated anti-CD40 antibody mixtures bind to separate epitopes located on CRD1 and CRD3 of CD40 ECD. Antibody 17303 binds an epitope on CRD1 distant from that of the selicrelumab analogue, whereas antibodies 16040 and 15833 bind to closely related epitopes located at CRD3 of CD40.

1-42. (canceled)

43. An anti-CD40 antibody or an antigen-binding portion thereof, wherein the antibody binds to the same epitope of human CD40 as an antibody comprising:
a) a heavy chain (HC) comprising the amino acid sequences of SEQ ID NOs: 3 and 61 and a light chain (LC) comprising the amino acid sequences of SEQ ID NOs: 4 and 62;
b) an HC comprising the amino acid sequences of SEQ ID NOs: 13 and 61 and an LC comprising the amino acid sequences of SEQ ID NOs: 14 and 62;
c) an HC comprising the amino acid sequences of SEQ ID NOs: 23 and 61 and an LC comprising the amino acid sequences of SEQ ID NOs: 24 and 62;
d) an HC comprising the amino acid sequences of SEQ ID NOs: 33 and 61 and an LC comprising the amino acid sequences of SEQ ID NOs: 34 and 62;
e) an HC comprising the amino acid sequences of SEQ ID NOs: 43 and 61 and an LC comprising the amino acid sequences of SEQ ID NOs: 44 and 62; or
f) an HC comprising the amino acid sequences of SEQ ID NOs: 53 and 61 and an LC comprising the amino acid sequences of SEQ ID NOs: 54 and 62.

44. An anti-CD40 antibody or an antigen-binding portion thereof, wherein said antibody comprises the H-CDR1-3 and L-CDR1-3 amino acid sequences of:
a) SEQ ID NOs: 5-10, respectively;
b) SEQ ID NOs: 15-20, respectively;
c) SEQ ID NOs: 25-30, respectively;
d) SEQ ID NOs: 35-40, respectively;
e) SEQ ID NOs: 45-50, respectively; or
f) SEQ ID NOs: 55-60, respectively.

45. The anti-CD40 antibody or antigen-binding portion of claim 44, wherein said antibody comprises a heavy chain variable domain and a light chain variable domain comprising the amino acid sequences of:
a) SEQ ID NOs: 3 and 4, respectively;
b) SEQ ID NOs: 13 and 14, respectively;
c) SEQ ID NOs: 23 and 24, respectively;
d) SEQ ID NOs: 33 and 34, respectively;
e) SEQ ID NOs: 43 and 44, respectively; or
f) SEQ ID NOs: 53 and 54, respectively.

46. The anti-CD40 antibody or antigen-binding portion of claim 44, wherein the antibody or antigen-binding portion has at least one property selected from:
a) binds to human CD40 with a K_{D} of 8×10⁻⁹M or less;
b) binds to cynomolgus CD40 with a K_{D} of 6×10⁻⁸ M or less;
c) stimulates CD40 activity in a CD40-NFkB-luciferase reporter assay;
d) does not inhibit CD40 ligand activity in a CD40-NFkB-luciferase reporter assay; and
e) stimulates proliferation of B cells in vitro in the presence of IL-21.

47. The anti-CD40 antibody of claim 44, comprising:
a) a heavy chain (HC) comprising the amino acid sequences of SEQ ID NOs: 3 and 61 and a light chain (LC) comprising the amino acid sequences of SEQ ID NOs: 4 and 62;
b) an HC comprising the amino acid sequences of SEQ ID NOs: 13 and 61 and an LC comprising the amino acid sequences of SEQ ID NOs: 14 and 62;
c) an HC comprising the amino acid sequences of SEQ ID NOs: 23 and 61 and an LC comprising the amino acid sequences of SEQ ID NOs: 24 and 62;
d) an HC comprising the amino acid sequences of SEQ ID NOs: 33 and 61 and an LC comprising the amino acid sequences of SEQ ID NOs: 34 and 62;
e) an HC comprising the amino acid sequences of SEQ ID NOs: 43 and 61 and an LC comprising the amino acid sequences of SEQ ID NOs: 44 and 62; or
f) an HC comprising the amino acid sequences of SEQ ID NOs: 53 and 61 and an LC comprising the amino acid sequences of SEQ ID NOs: 54 and 62.

48. A composition comprising a first anti-CD40 antibody or an antigen-binding portion thereof, and a second, distinct anti-CD40 antibody or an antigen-binding portion thereof, comprising:
a) a first antibody or antigen-binding portion thereof that comprises H-CDR1-3 and L-CDR1-3 amino acid sequences of SEQ ID NOs: 5-10, respectively; and a second antibody or antigen-binding portion thereof that comprises H-CDR1-3 and L-CDR1-3 amino acid sequences of SEQ ID NOs: 15-20, respectively;
b) a first antibody or antigen-binding portion thereof that comprises H-CDR1-3 and L-CDR1-3 amino acid sequences of SEQ ID NOs: 5-10, respectively; and a second antibody or antigen-binding portion thereof that comprises H-CDR1-3 and L-CDR1-3 amino acid sequences of SEQ ID NOs: 25-30, respectively;
c) a first antibody or antigen-binding portion thereof that comprises H-CDR1-3 and L-CDR1-3 amino acid sequences of SEQ ID NOs: 5-10, respectively; and a second antibody or antigen-binding portion thereof that comprises H-CDR1-3 and L-CDR1-3 amino acid sequences of SEQ ID NOs: 55-60, respectively;
d) a first antibody or antigen-binding portion thereof that comprises H-CDR1-3 and L-CDR1-3 amino acid sequences of SEQ ID NOs: 15-20, respectively; and a second antibody or antigen-binding portion thereof that comprises H-CDR1-3 and L-CDR1-3 amino acid sequences of SEQ ID NOs: 45-50, respectively; or
e) a first antibody or antigen-binding portion thereof that comprises H-CDR1-3 and L-CDR1-3 amino acid sequences of SEQ ID NOs: 15-20, respectively; and a second antibody or antigen-binding portion thereof that comprises H-CDR1-3 and L-CDR1-3 amino acid sequences of SEQ ID NOs: 35-40, respectively.

49. The composition of claim 48, comprising:
a) a first antibody or antigen-binding portion thereof that comprises VH and VL amino acid sequences of SEQ ID NOs: 3 and 4, respectively; and a second antibody or antigen-binding portion thereof that comprises VH and VL amino acid sequences of SEQ ID NOs: 13 and 14, respectively;
b) a first antibody or antigen-binding portion thereof that comprises VH and VL amino acid sequences of SEQ ID NOs: 3 and 4, respectively; and a second antibody or antigen-binding portion thereof that comprises VH and VL amino acid sequences of SEQ ID NOs: 23 and 24, respectively;
c) a first antibody or antigen-binding portion thereof that comprises VH and VL amino acid sequences of SEQ ID NOs: 3 and 4, respectively; and a second antibody or antigen-binding portion thereof that comprises VH and VL amino acid sequences of SEQ ID NOs: 53 and 54, respectively;
d) a first antibody or antigen-binding portion thereof that comprises VH and VL amino acid sequences of SEQ ID NOs: 13 and 14, respectively; and a second antibody or antigen-binding portion thereof that comprises VH and VL amino acid sequences of SEQ ID NOs: 43 and 44, respectively; or
e)- a first antibody or antigen-binding portion thereof that comprises VH and VL amino acid sequences of SEQ ID NOs: 13 and 14, respectively; and a second antibody or antigen-binding portion thereof that comprises VH and VL amino acid sequences of SEQ ID NOs: 33 and 34, respectively.

50. The composition of claim 48, comprising:
a) a first antibody or antigen-binding portion thereof that comprises an HC comprising the amino acid sequences of SEQ ID NOs: 3 and 61 and an LC comprising the amino acid sequences of SEQ ID NOs: 4 and 62; and a second antibody or antigen-binding portion thereof that comprises an HC comprising the amino acid sequences of SEQ ID NOs: 13 and 61 and an LC comprising the amino acid sequences of SEQ ID NOs: 14 and 62;
b) a first antibody or antigen-binding portion thereof that comprises an HC comprising the amino acid sequences of SEQ ID NOs: 3 and 61 and an LC comprising the amino acid sequences of SEQ ID NOs: 4 and 62; and a second antibody or antigen-binding portion thereof that comprises an HC comprising the amino acid sequences of SEQ ID NOs: 23 and 61 and an LC comprising the amino acid sequences of SEQ ID NOs: 24 and 62;
c) a first antibody or antigen-binding portion thereof that comprises an HC comprising the amino acid sequences of SEQ ID NOs: 3 and 61 and an LC comprising the amino acid sequences of SEQ ID NOs: 4 and 62; and a second antibody or antigen-binding portion thereof that comprises an HC comprising the amino acid sequences of SEQ ID NOs: 53 and 61 and an LC comprising the amino acid sequences of SEQ ID NOs: 54 and 62;
d) a first antibody or antigen-binding portion thereof that comprises an HC comprising the amino acid sequences of SEQ ID NOs: 13 and 61 and an LC comprising the amino acid sequences of SEQ ID NOs: 14 and 62; and a second antibody or antigen-binding portion thereof that comprises an HC comprising the amino acid sequences of SEQ ID NOs: 43 and 61 and an LC comprising the amino acid sequences of SEQ ID NOs: 44 and 62; or
e) a first antibody or antigen-binding portion thereof that comprises an HC comprising the amino acid sequences of SEQ ID NOs: 13 and 61 and an LC comprising the amino acid sequences of SEQ ID NOs: 14 and 62; and a second antibody or antigen-binding portion thereof that comprises an HC comprising the amino acid sequences of SEQ ID NOs: 33 and 61 and an LC comprising the amino acid sequences of SEQ ID NOs: 34 and 62.

51. The composition of claim 48, wherein the composition has at least one property selected from:
a) stimulates CD40 activity in a CD40-NFkB-luciferase reporter assay;
b) does not inhibit CD40 ligand activity in a CD40-NFkB-luciferase reporter assay;
c) stimulates CD40 activity in a one-way mixed lymphocyte reaction (MLR) assay;
d) stimulates proliferation of B cells in vitro in the presence of IL-21;
e) stimulates dendritic cell activation in vitro;
f) stimulates proliferation of B cells from splenocytes;
g) inhibits tumor growth in vivo in huCD34-NOG mice engrafted with Raji cells; and
h) inhibits tumor growth in vivo in CD40 HuGEMM mice engrafted with MC38-OVA colon carcinoma cells.

52. A pharmaceutical composition comprising the anti-CD40 antibody or antigen-binding portion of claim 43.

53. An isolated nucleic acid molecule comprising a nucleotide sequence that encodes the heavy chain or an antigen-binding portion thereof, or a nucleotide sequence that encodes the light chain or an antigen-binding portion thereof, or both, of the anti-CD40 antibody or antigen-binding portion of claim 43.

54. A vector comprising the isolated nucleic acid molecule of claim 53, wherein said vector further comprises an expression control sequence.

55. A host cell comprising a nucleotide sequence that encodes the heavy chain or an antigen-binding portion thereof, and a nucleotide sequence that encodes the light chain or an antigen-binding portion thereof, of the anti-CD40 antibody or antigen-binding portion of claim 43.

56. A method for producing an anti-CD40 antibody or an antigen-binding portion thereof, comprising providing a host cell according to claim 55, culturing said host cell under conditions suitable for expression of the antibody or portion, and isolating the resulting antibody or portion.

57. A method for producing the anti-CD40 antibody composition of claim 48, comprising providing a first anti-CD40 antibody or antigen-binding portion and a second anti-CD40 antibody or antigen-binding portion, and admixing the two antibodies or portions.

58. A bi-specific binding molecule comprising the antigen-binding portion of one or two distinct anti-CD40 antibodies according to claim 43.

59. A method for treating cancer in a patient,
treating an immune disorder in a patient in need thereof, or
enhancing immune activity in a patient in need thereof,
comprising administering to said patient a therapeutically effective amount of the anti-CD40 antibody or antigen-binding portion of claim 43.

60. A method for treating cancer in a patient,
treating an immune disorder in a patient in need thereof, or
enhancing immune activity in a patient in need thereof,
comprising administering to said patient a therapeutically effective amount of the composition of claim 48.

61. The method of claim 60, wherein the cancer is melanoma, head and neck cancer, esophageal cancer, non-small cell lung cancer, breast cancer, ovarian cancer, pancreatic cancer, urothelial carcinoma, renal cancer, colon cancer, prostate cancer, epithelial cancer, mesothelioma, sarcoma, non-Hodgkin's lymphoma, B cell lymphoma, diffuse large B cell lymphoma, follicular lymphoma, chronic lymphocytic leukemia, or multiple myeloma.

62. The method of claim 60, further comprising administering to the patient an immunostimulatory agent, a vaccine, a chemotherapeutic agent, an anti-neoplastic agent, an anti-angiogenic agent, a tyrosine kinase inhibitor, or radiation therapy.
